(19) United States
(12) Patent Application Publication
(10) Pub. No.: US 2022/0262468 A1
(43) Pub. Date: Aug. 18, 2022

(54) System and method for managing electronic medical records

(71) Applicant: Noor AlQabandi, Boston, MA (US)

(72) Inventor: Noor AlQabandi, Boston, MA (US)

(21) Appl. No.: 17/673,509

(22) Filed: Feb. 16, 2022

Related U.S. Application Data
(60) Provisional application No. 63/149,923, filed on Feb. 16, 2021.

(51) Int. Cl. G16H 10/60; G16H 80/00; G06N 20/00

(52) U.S. Cl. CPC G16H 10/60; G06N 20/00; G16H 80/00

(57) ABSTRACT

A system and method for managing electronic medical/dental records are provided. The system is designed to improve communication and increase medical accessibility in a convenient way for both medical/dental practitioners and patients, empower patients and provide secure communication, increase access to medical care and decrease time and cost associated with medical consultations.

### TECHNICAL FIELD

The present invention relates to a system and computer-implemented methods for managing electronic medical records and medical record information. More specifically, the present invention relates to a system and computer-implement methods for providing a communication platform for medical providers and patients.

### BACKGROUND ART

Access to electronic health records by the patient anytime is still an issue in the United States. Patients go through the traditional way using phone calls, emails, sent by fax, or collect in office. Teledentistry became more popular than before. Jobs that depend on travel and cost of time are increasing in the United States. After going over the main health problems, persona assessment and analysis, public survey and competitive analysis. An innovative mobile health design has been created as a solution to improve communication and increase dental accessibility in easy and organized between patients and dentists.

Health records and history taking is a necessity to provide patients with adequate diagnosis and treatment. The value and awareness of collecting health data have dramatically increased in this decade, along with the advanced technology and security. Governments and medical health centers have implemented Electronic Health Record (EHR) system to improve care quality and management among their population. Even though the advanced level of Health care system and delivery that United State have reached, they still lack Electronic Health Record Network System (EHRNS) at the federal, state, and individual levels that allow the patient to access in time of need.

EHR is a digital record system that can provide comprehensive health information that contains; patient's medical and dental history, vital signs, prescribed medication along with dosage and duration, allergies, radiographs, lab and tests results, immunization dates, progress notes, patient's demographics and administrative and billing data. It has been created to improve patient care among medical providers, pharmacies, emergency facilities, school and workplace clinics.

Tele dentistry means that dental professionals communicate with patients through virtual calls to provide quick consultation or guidance over long distances. This concept is used in all medical health that they provide medical or dental consultation to their family, friends, and patients. Studies have shown the positive effect of this concept in increasing dental accessibility, reduce time and cost regarding dental consultation for both patients and dentists. With the modern advance of technology, mainly in smartphones, video calls are commonly used to ease communication and understanding. COVID 19 crises have heightened this method, and more dentists started to use it in their practices.

With the accelerate development of EHS systems, increasing the number of Dental clinics using EHR increases the number of jobs supported by travel, globalization, and growing generation utilizing the enhanced technology communication in other industries. Also, the increase in public health demands of quality treatments and efficient communications with the dentist. Are all directing and pushing the dental sector to use entrepreneurship intervention in developing traditional EHR and improve communication.

Therefore, there is a need for an efficient and convenient system that is designed to improve communication and increase medical accessibility in a convenient way for both medical practitioners (such as doctors or dentists, for example) and patients, empower patients and provide secure communication, increase access to medical care and decrease time and cost associated with medical consultations.

### SUMMARY OF THE EMBODIMENTS

The present invention provides an innovative software-based platform that has been created as a solution to improve communications and increase medical (or dental) accessibility in a convenient way for both doctors (or dentists) and patients. It includes two interlinked mobile platforms: one for patients, and the other for dentist. The platforms are comprehensive and contain a plurality of features, allowing patients to find dentists/lab technicians and providing dentists with means to promote their name/clinic with additional functional dental care-related features.

The system of the present invention is designed to enable faster access to patient records for more coordinated and efficient care; to empower the patient to understand their own oral health in easy friendly design; to improve and maintain the patient's behavior health towards healthier smile by direct notifications; to support caregiver to manage and keep track on their career's oral health; to increase the preventive oral care and reduce eventually the cost for dental treatments; to securely share electronic information with patients and dentists; and to provide useful, convenient, and coordinated audio or virtual consultations scheduling initial appointments. The system is further designed to aid providers in effective diagnosis of patients, reduction of medical errors and repetitive examinations, and providing safer and more comprehensive care; to increase patient access to dental care with a compatible dentist (in terms of location, insurance coverage, and dental treatment); to increase efficiency in patient tracking through referrals between various dentists; and to provide a nationwide platform that enables patients to find dentists and schedule appointments directly by delivery of electronic dental and health record (EDR/EHR) prior to initial appointment.

Other aspects, embodiments and features of the system and method will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed device and method, will be better understood when read in conjunction with the attached drawings. It should be understood, however, that neither the device nor the method is limited to the precise arrangements and instrumentalities shown.

FIG. 1 is a block diagram depicting an example of a computing device as described herein.

FIG. 2 is a block diagram depicting an example of a network-based platform, as described herein.

FIG. 3 is a schematic diagram showing the general architecture of the system in accordance with an embodiment of the present invention.

FIGS. 4A-4Y illustrate the functionalities of the patient platform in accordance with the embodiments of the present invention.

FIGS. 5A-5L illustrate the functionalities of the dentist platform in accordance with the embodiments of the present invention.

FIG. 6 is a schematic diagram illustrating a process flow associated with five main features of the patient platform.

FIG. 7 is a process flow for estimating costs section of the patient platform.

FIG. 8 is a flow diagram illustrating a method of registering a family member using the patient platform.

FIG. 9 is a flow chart illustrating a process flow of the functionalities of the dentist platform in accordance with an embodiment of the present invention.

FIG. 10 is an illustration of the process flow for the feedback and consent forms sections of the dentist platform.

FIG. 11 shows the multidisciplinary team feature for referring patient for endodontic treatment and orthodontic work.

### DETAILED DESCRIPTION OF SPECIFIC EMBODIMENTS

The system of the present invention is designed to provide innovative modern secured communication through a cloud-based system using mobile health design software-based application.

Some embodiments of the disclosed system and methods will be better understood by reference to the following comments concerning computing devices. A “computing device” 100 may be defined as including personal computers, laptops, tablets, smart phones, and any other computing device capable of supporting an application as described herein. The system and method disclosed herein will be better understood in light of the following observations concerning the computing devices that support the disclosed application and concerning the nature of web applications in general.

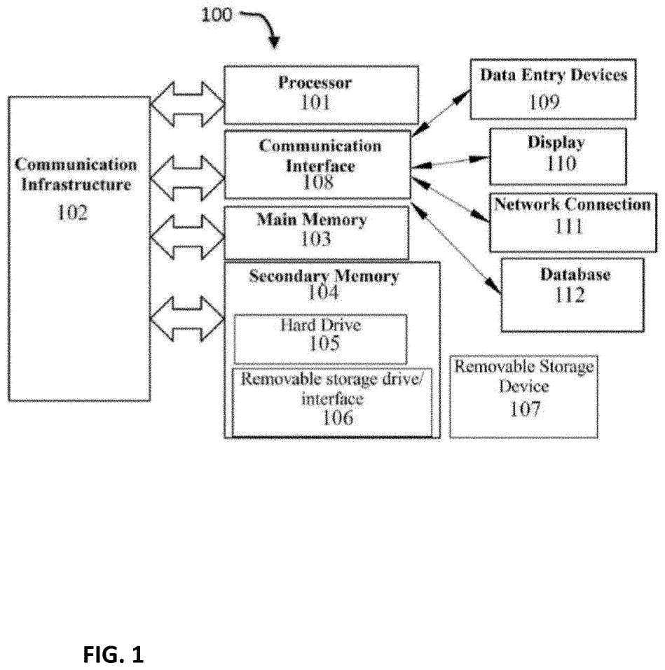

Referring now to the drawings in detail. An exemplary computing device is illustrated by FIG. 1. The processor 101 may be a special purpose or a general-purpose processor device. As will be appreciated by persons skilled in the relevant art, the processor device 101 may also be a single processor in a multi-core/multiprocessor system, such system operating alone, or in a cluster of computing devices operating in a cluster or server farm. The processor 101 is connected to a communication infrastructure 102, for example, a bus, message queue, network, or multi-core message-passing scheme.

The computing device also includes a main memory 103, such as random access memory (RAM), and may also include a secondary memory 104. Secondary memory 104 may include, for example, a hard disk drive 105, a removable storage drive or interface 106, connected to a removable storage unit 107, or other similar means. As will be appreciated by persons skilled in the relevant art, a removable storage unit 107 includes a computer usable storage medium having stored therein computer software and/or data. Examples of additional means creating secondary memory 104 may include a program cartridge and cartridge interface (such as that found in video game devices), a removable memory chip (such as an EPROM, or PROM) and associated socket, and other removable storage units 107 and interfaces 106 which allow software and data to be transferred from the removable storage unit 107 to the computer system. In some embodiments, to “maintain” data in the memory of a computing device means to store that data in that memory in a form convenient for retrieval as required by the algorithm at issue, and to retrieve, update, or delete the data as needed.

The computing device may also include a communications interface 108.

The communications interface 108 allows software and data to be transferred between the computing device and external devices. The communications interface 108 may include a modem, a network interface (such as an Ethernet card), a communications port, a PCMCIA slot and card, or other means to couple the computing device to external devices. Software and data transferred via the communications interface 108 may be in the form of signals, which may be electronic, electromagnetic, optical, or other signals capable of being received by the communications interface 108. These signals may be provided to the communications interface 108 via wire or cable, fiber optics, a phone line, a cellular phone link, and radio frequency link or other communications channels. Other devices may be coupled to the computing device 100 via the communications interface 108. In some embodiments, a device or component is “coupled” to a computing device 100 if it is so related to that device that the product or means and the device may be operated together as one machine. In particular, a piece of electronic equipment is coupled to a computing device if it is incorporated in the computing device (e.g. a built-in camera on a smart phone), attached to the device by wires capable of propagating signals between the equipment and the device (e.g. a mouse connected to a personal computer by means of a wire plugged into one of the computer's ports), tethered to the device by wireless technology that replaces the ability of wires to propagate signals (e.g. a wireless BLUETOOTH® headset for a mobile phone), or related to the computing device by shared membership in some network consisting of wireless and wired connections between multiple machines (e.g. a printer in an office that prints documents to computers belonging to that office, no matter where they are, so long as they and the printer can connect to the internet). A computing device 100 may be coupled to a second computing device (not shown); for instance, a server may be coupled to a client device, as described below in greater detail.

The communications interface in the system embodiments discussed herein facilitates the coupling of the computing device with data entry devices 109, the device's display 110, and network connections, whether wired or wireless 111. In some embodiments, “data entry devices” 109 are any equipment coupled to a computing device that may be used to enter data into that device. This definition includes, without limitation, keyboards, computer mice, touchscreens, digital cameras, digital video cameras, wireless antennas, Global Positioning System devices, audio input and output devices, gyroscopic orientation sensors, proximity sensors, compasses, scanners, specialized reading devices such as fingerprint or retinal scanners, and any hardware device capable of sensing electromagnetic radiation, electromagnetic fields, gravitational force, electromagnetic force, temperature, vibration, or pressure. A computing device's “manual data entry devices” is the set of all data entry devices coupled to the computing device that permit the user to enter data into the computing device using manual manipulation. Manual entry devices include without limitation keyboards, keypads, touchscreens, track-pads, computer mice, buttons, and other similar components. A computing device may also possess a navigation facility. The computing device's “navigation facility” may be any facility coupled to the computing device that enables the device accurately to calculate the device's location on the surface of the Earth. Navigation facilities can include a receiver configured to communicate with the Global Positioning System or with similar satellite networks, as well as any other system that mobile phones or other devices use to ascertain their location, for example by communicating with cell towers. In some embodiments, a computing device's “display” 109 is a device coupled to the computing device, by means of which the computing device can display images. Display includes without limitation monitors, screens, television devices, and projectors.

Computer programs (also called computer control logic) are stored in main memory 103 and/or secondary memory 104. Computer programs may also be received via the communications interface 108. Such computer programs, when executed, enable the processor device 101 to implement the system embodiments discussed below. Accordingly, such computer programs represent controllers of the system. Where embodiments are implemented using software, the software may be stored in a computer program product and loaded into the computing device using a removable storage drive or interface 106, a hard disk drive 105, or a communications interface 108.

Computer program code for carrying out operations of the present invention may be written in an object oriented programming language such as, but not limited to, Java, Smalltalk or C++. However, the computer program code for carrying out operations of the present invention may also be written in conventional procedural programming languages such as the “C” programming language. Computer readable program instructions for carrying out operations of the present invention may also be assembler instructions, instruction-set-architecture (ISA) instructions, machine instructions, machine dependent instructions, microcode, firmware instructions, state-setting data, or either source code or object code written in any combination of one or more programming languages described above. In some instances, the computer readable program can be executed in the reverse order, depending upon the functionality involved. It will also be noted that each block of the flowchart illustrations, and combinations of blocks in the flowchart illustrations, can be implement by special purpose hardware-based systems that perform the specified functions or acts or carry out combinations of special purpose hardware and computer instructions.

The computing device may also store data in database 112 accessible to the device. A database 112 is any structured collection of data. As used herein, databases can include “NoSQL” data stores, which store data in a few key-value structures such as arrays for rapid retrieval using a known set of keys (e.g. array indices). Another possibility is a relational database, which can divide the data stored into fields representing useful categories of data. As a result, a stored data record can be quickly retrieved using any known portion of the data that has been stored in that record by searching within that known datum's category within the database 112, and can be accessed by more complex queries, using languages such as Structured Query Language, which retrieve data based on limiting values passed as parameters and relationships between the data being retrieved. More specialized queries, such as image matching queries, may also be used to search some databases. A database can be created in any digital memory.

Persons skilled in the relevant art will also be aware that while any computing device must necessarily include facilities to perform the functions of a processor 101, a communication infrastructure 102, at least a main memory 103, and usually a communications interface 108, not all devices will necessarily house these facilities separately. For instance, in some forms of computing devices as defined above, processing 101 and memory 103 could be distributed through the same hardware device, as in a neural net, and thus the communications infrastructure 102 could be a property of the configuration of that particular hardware device. Many devices do practice a physical division of tasks as set forth above, however, and practitioners skilled in the art will understand the conceptual separation of tasks as applicable even where physical components are merged.

The computing device 100 may employ one or more security measures to protect the computing device 100 or its data. For instance, the computing device 100 may protect data using a cryptographic system. In one embodiment, a cryptographic system is a system that converts data from a first form, known as “plaintext,” which is intelligible when viewed in its intended format, into a second form, known as “cyphertext,” which is not intelligible when viewed in the same way. The cyphertext may be unintelligible in any format unless first converted back to plaintext. In one embodiment, the process of converting plaintext into cyphertext is known as “encryption.” The encryption process may involve the use of a datum, known as an “encryption key,” to alter the plaintext. The cryptographic system may also convert cyphertext back into plaintext, which is a process known as “decryption.” The decryption process may involve the use of a datum, known as a “decryption key,” to return the cyphertext to its original plaintext form. In embodiments of cryptographic systems that are “symmetric,” the decryption key is essentially the same as the encryption key: possession of either key makes it possible to deduce the other key quickly without further secret knowledge. The encryption and decryption keys in symmetric cryptographic systems may be kept secret, and shared only with persons or entities that the user of the cryptographic system wishes to be able to decrypt the cyphertext. One example of a symmetric cryptographic system is the Advanced Encryption Standard (“AES”), which arranges plaintext into matrices and then modifies the matrices through repeated permutations and arithmetic operations with an encryption key.

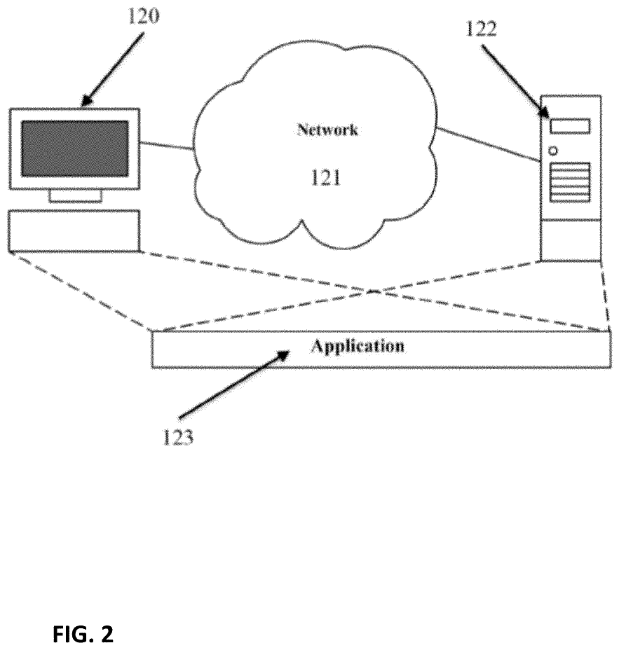

The systems may be deployed in a number of ways, including on a stand-alone computing device, a set of computing devices working together in a network, or a web application. Persons of ordinary skill in the art will recognize a web application as a particular kind of computer program system designed to function across a network, such as the Internet. A schematic illustration of a web application platform is provided in FIG. 2. Web application platforms typically include at least one client device 120, which is a computing device as described above. The client device 120 connects via some form of network connection to a network 121, such as the Internet. The network 121 may be any arrangement that links together computing devices 120, 122, and includes without limitation local and international wired networks including telephone, cable, and fiber-optic networks, wireless networks that exchange information using signals of electromagnetic radiation, including cellular communication and data networks, and any combination of those wired and wireless networks. Also connected to the network 121 is at least one server 122, which is also a computing device as described above, or a set of computing devices that communicate with each other and work in concert by local or network connections. Of course, practitioners of ordinary skill in the relevant art will recognize that a web application can, and typically does, run on several servers 122 and a vast and continuously changing population of client devices 120. Computer programs on both the client device 120 and the server 122 configure both devices to perform the functions required of the web application 123. Web applications 123 can be designed so that the bulk of their processing tasks are accomplished by the server 122, as configured to perform those tasks by its web application program, or alternatively by the client device 120. Some web applications 123 are designed so that the client device 120 solely displays content that is sent to it by the server 122, and the server 122 performs all of the processing, business logic, and data storage tasks. Such “thin client” web applications are sometimes referred to as “cloud” applications, because essentially all computing tasks are performed by a set of servers 122 and data centers visible to the client only as a single opaque entity, often represented on diagrams as a cloud.

Many computing devices, as defined herein, come equipped with a specialized program, known as a web browser, which enables them to act as a client device 120 at least for the purposes of receiving and displaying data output by the server 122 without any additional programming. Web browsers can also act as a platform to run so much of a web application as is being performed by the client device 120, and it is a common practice to write the portion of a web application calculated to run on the client device 120 to be operated entirely by a web browser. Such browser-executed programs are referred to herein as “client-side programs,” and frequently are loaded onto the browser from the server 122 at the same time as the other content the server 122 sends to the browser. However, it is also possible to write programs that do not run on web browsers but still cause a computing device to operate as a web application client 120. Thus, as a general matter, web applications 123 require some computer program configuration of both the client device (or devices) 120 and the server 122. The computer program that comprises the web application component on either computing device's system FIG. 1 configures that device's processor 200 to perform the portion of the overall web application's functions that the programmer chooses to assign to that device. Persons of ordinary skill in the art will appreciate that the programming tasks assigned to one device may overlap with those assigned to another, in the interests of robustness, flexibility, or performance. Furthermore, although the best known example of a web application as used herein uses the kind of hypertext markup language protocol popularized by the World Wide Web, practitioners of ordinary skill in the art will be aware of other network communication protocols, such as File Transfer Protocol, that also support web applications as defined herein.

The one or more client devices 120 and the one or more servers 122 may communicate using any protocol according to which data may be transmitted from the client 120 to the server 122 and vice versa. As a non-limiting example, the client 120 and server 122 may exchange data using the Internet protocol suite, which includes the transfer control protocol (TCP) and the Internet Protocol (IP), and is sometimes referred to as TCP/IP. In some embodiments, the client and server 122 encrypt data prior to exchanging the data, using a cryptographic system as described above. In one embodiment, the client 120 and server 122 exchange the data using public key cryptography; for instance, the client and the server 122 may each generate a public and private key, exchange public keys, and encrypt the data using each others' public keys while decrypting it using each others' private keys.

In some embodiments, the client 120 authenticates the server 122 or vice-versa using digital certificates. In one embodiment, a digital certificate is a file that conveys information and links the conveyed information to a “certificate authority” that is the issuer of a public key in a public key cryptographic system. The certificate in some embodiments contains data conveying the certificate authority's authorization for the recipient to perform a task. The authorization may be the authorization to access a given datum. The authorization may be the authorization to access a given process. In some embodiments, the certificate may identify the certificate authority.

The linking may be performed by the formation of a digital signature. In one embodiment, a digital signature is an encrypted mathematical representation of a file using the private key of a public key cryptographic system. The signature may be verified by decrypting the encrypted mathematical representation using the corresponding public key and comparing the decrypted representation to a purported match that was not encrypted; if the signature protocol is well-designed and implemented correctly, this means the ability to create the digital signature is equivalent to possession of the private decryption key. Likewise, if the mathematical representation of the file is well-designed and implemented correctly, any alteration of the file will result in a mismatch with the digital signature; the mathematical representation may be produced using an alteration-sensitive, reliably reproducible algorithm, such as a hashing algorithm. A mathematical representation to which the signature may be compared may be included with the signature, for verification purposes; in other embodiments, the algorithm used to produce the mathematical representation is publicly available, permitting the easy reproduction of the mathematical representation corresponding to any file. In some embodiments, a third party known as a certificate authority is available to verify that the possessor of the private key is a particular entity; thus, if the certificate authority may be trusted, and the private key has not been stolen, the ability of an entity to produce a digital signature confirms the identity of the entity, and links the file to the entity in a verifiable way. The digital signature may be incorporated in a digital certificate, which is a document authenticating the entity possessing the private key by authority of the issuing certificate authority and signed with a digital signature created with that private key and a mathematical representation of the remainder of the certificate. In other embodiments, the digital signature is verified by comparing the digital signature to one known to have been created by the entity that purportedly signed the digital signature; for instance, if the public key that decrypts the known signature also decrypts the digital signature, the digital signature may be considered verified. The digital signature may also be used to verify that the file has not been altered since the formation of the digital signature.

The server 122 and client 120 may communicate using a security combining public key encryption, private key encryption, and digital certificates. For instance, the client 120 may authenticate the server 122 using a digital certificate provided by the server 122. The server 122 may authenticate the client 120 using a digital certificate provided by the client 120. After successful authentication, the device that received the digital certificate possesses a public key that corresponds to the private key of the device providing the digital certificate; the device that performed the authentication may then use the public key to convey a secret to the device that issued the certificate. The secret may be used as the basis to set up private key cryptographic communication between the client 120 and the server 122; for instance, the secret may be a private key for a private key cryptographic system. The secret may be a datum from which the private key may be derived. The client 120 and server 122 may then use that private key cryptographic system to exchange information until the secure communication protocol in which they are communicating ends. In some embodiments, this handshake and secure communication protocol is implemented using the secure sockets layer (SSL) protocol. In other embodiments, the protocol is implemented using the transport layer security (TLS) protocol. The server 122 and client 120 may communicate using hyper-text transfer protocol secure (HTTPS).

The primary task of the system of the present invention is to provide a summary of the patient's own dental record and allow the patient to request, access and exchange/upload their dental record in any place and time. It is configured to assist patients in finding a dentist according to their insurance and geographic compatibility. The user can designate a proxy and keep a track on their oral health. Telehealth consults can be requested in urgent and non-urgent settings with unparalleled convenience.

The system is further configured to allow dentists to track referred patient treatment records, follow up with patients, and communicate consents and other materials before the patient arrives at their clinic. The dentists will have a profile with rates and reviews from their patients. The system is also configured to provide a continued education courses for the registered dentists, where they can participate or promote dental seminars and conferences.

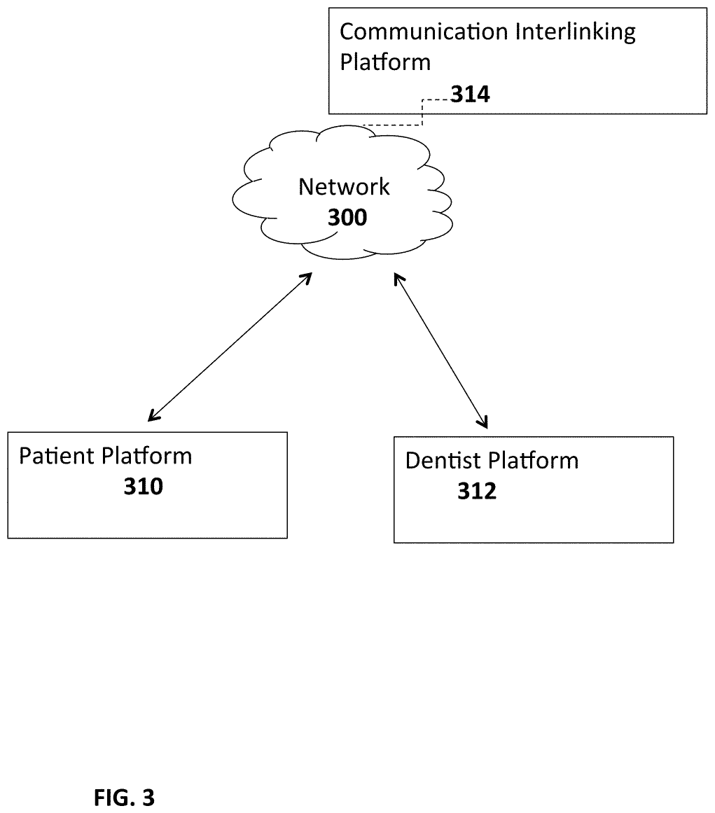

As illustrated in FIG. 3, the system consists of two main interlinked components: a patient platform 310 and a complementary dentist platform 312. The patient platform can be a mobile application (suitable for use on a smart phone configured with android or apple operating systems), whereas the dentist platform can be a website (in some instances, a website with React S and/or React Native framework) that can be accessed through both computers and smart mobile devices. In some instances, the dentist platform can be also a mobile application (including both android and apple operating systems). The system also includes a communication interlinking platform 314, which can be a separate website designed to communicate between the patient and dentist platforms through network 300. The system of the present disclosure is configured to be HIPPA compliant, secure, and capable of protecting user confidentiality.

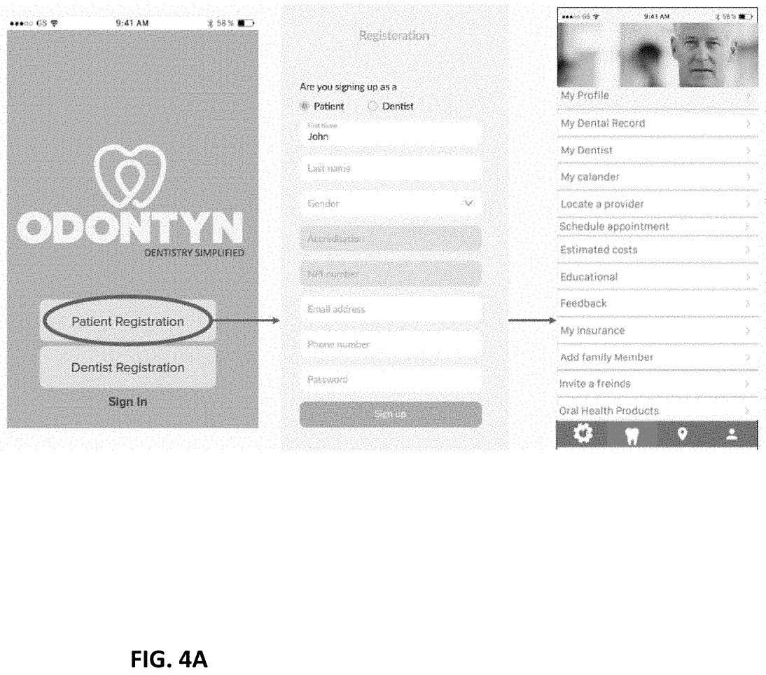

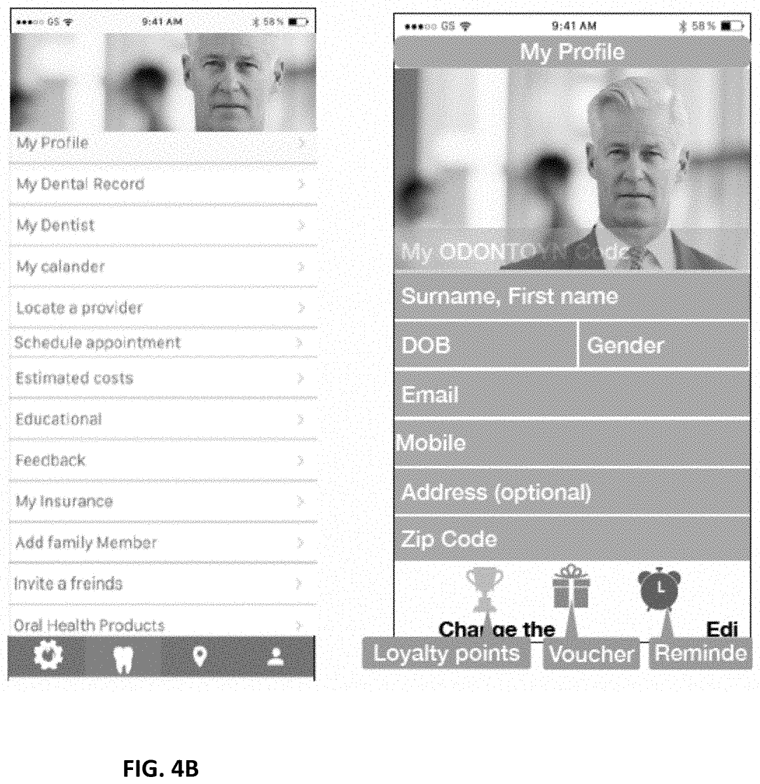

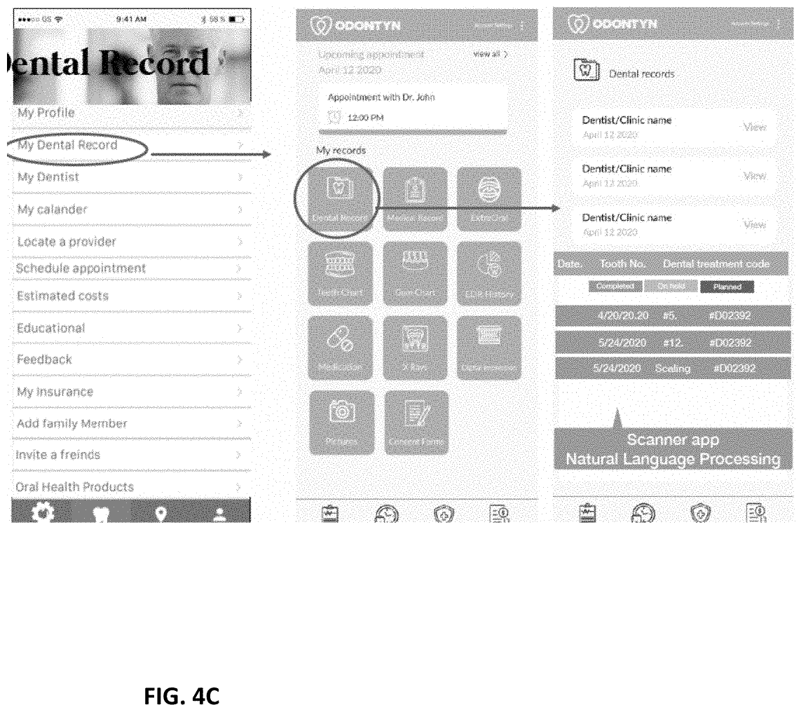

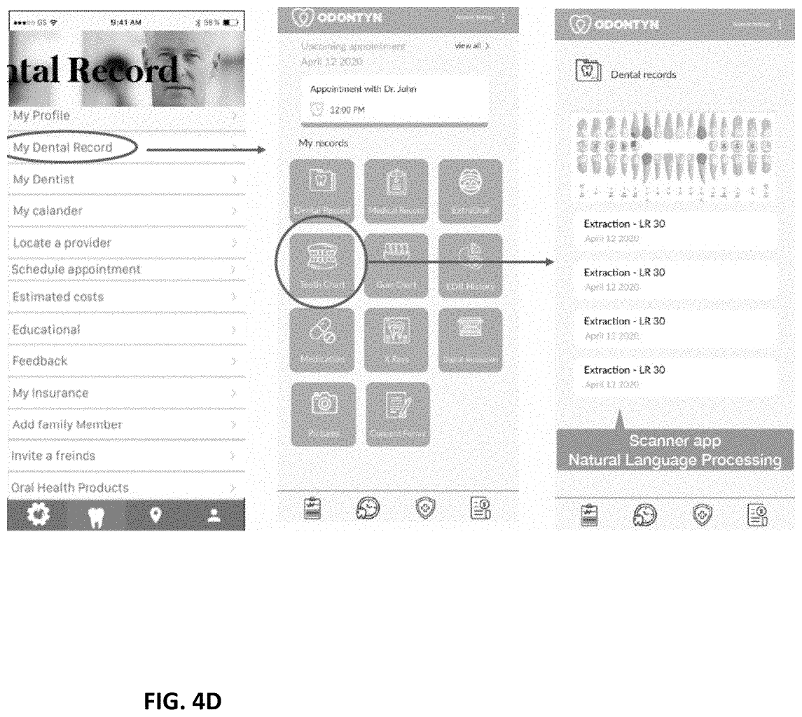

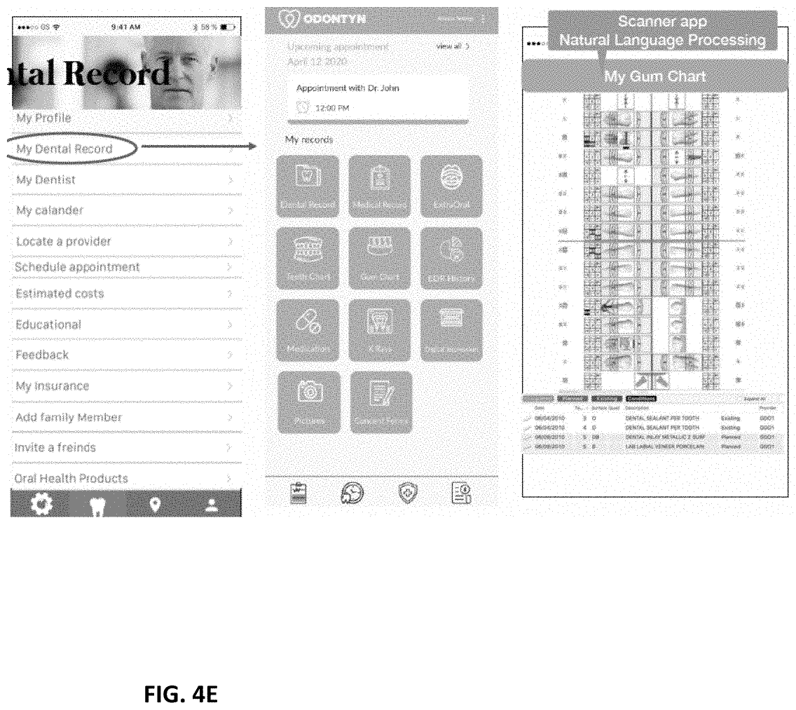

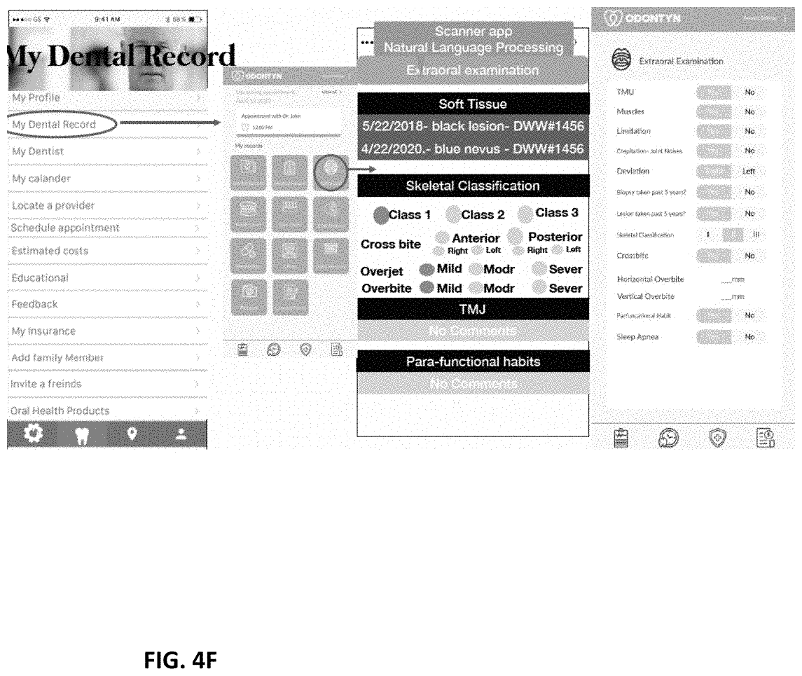

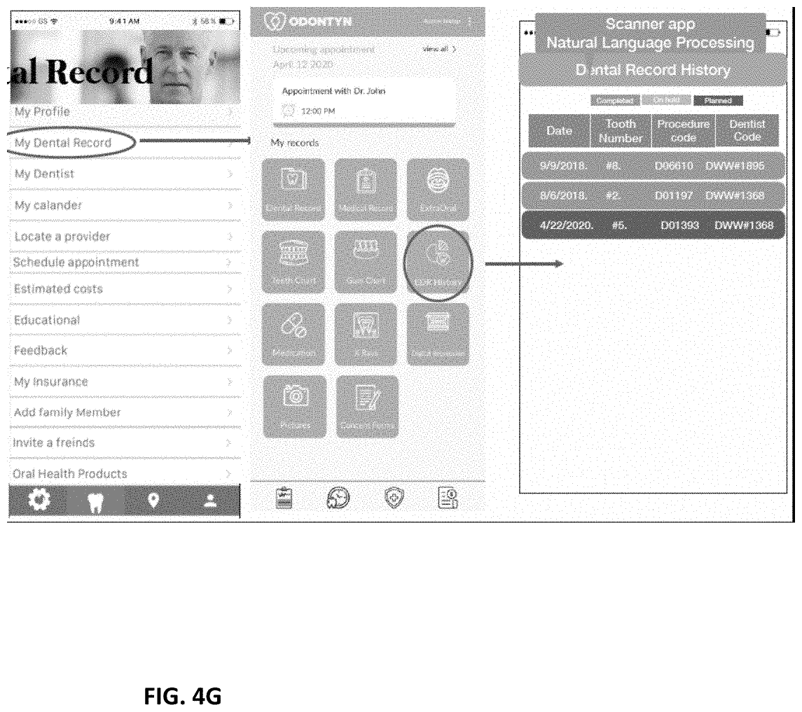

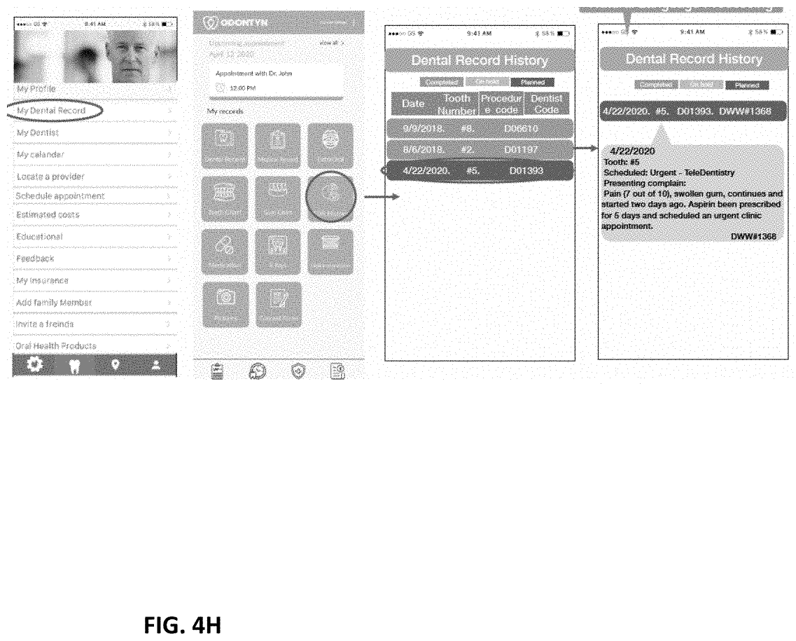

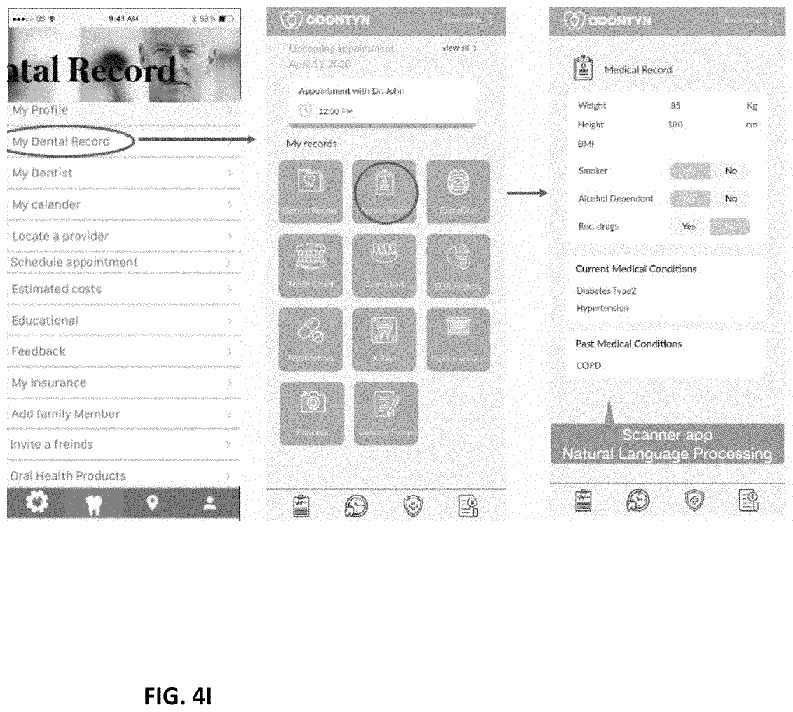

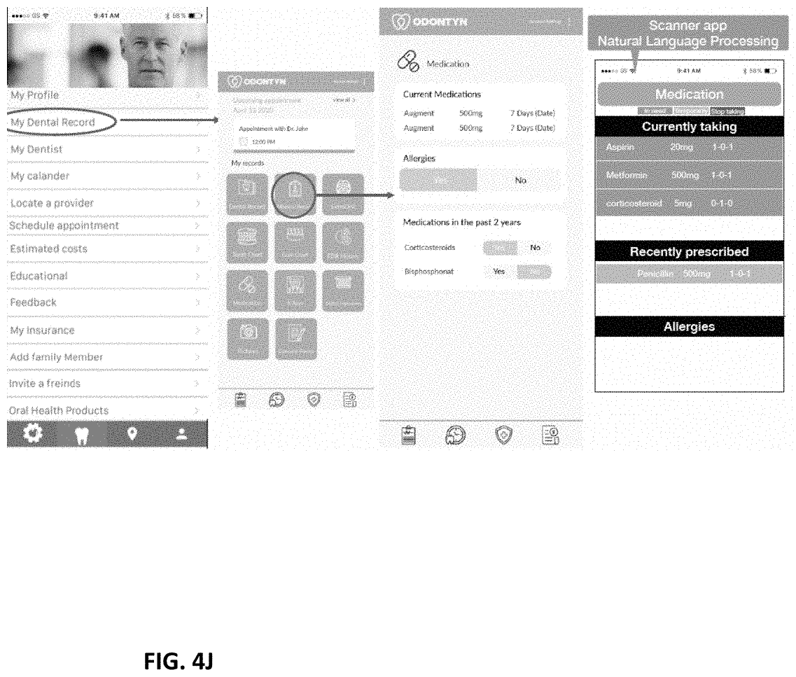

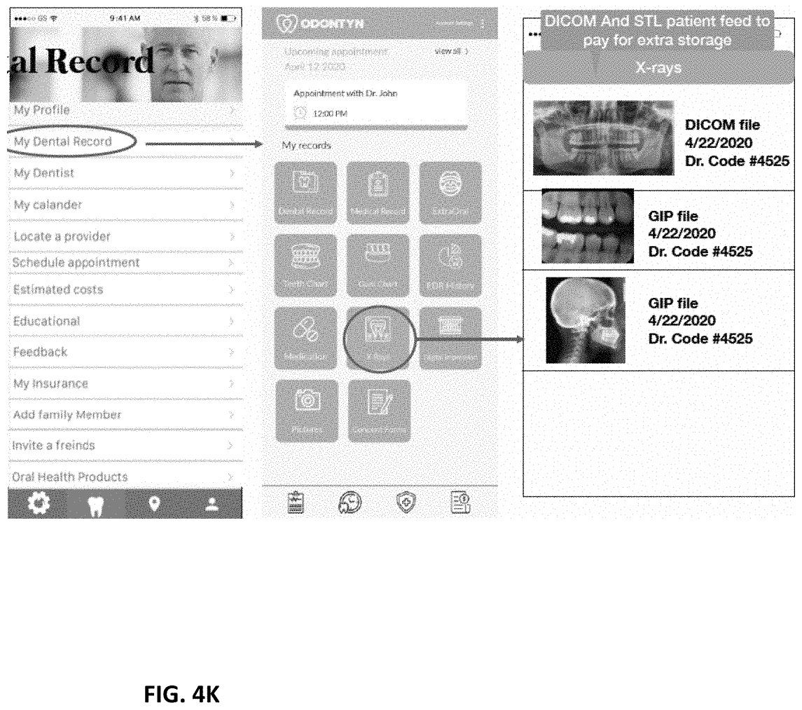

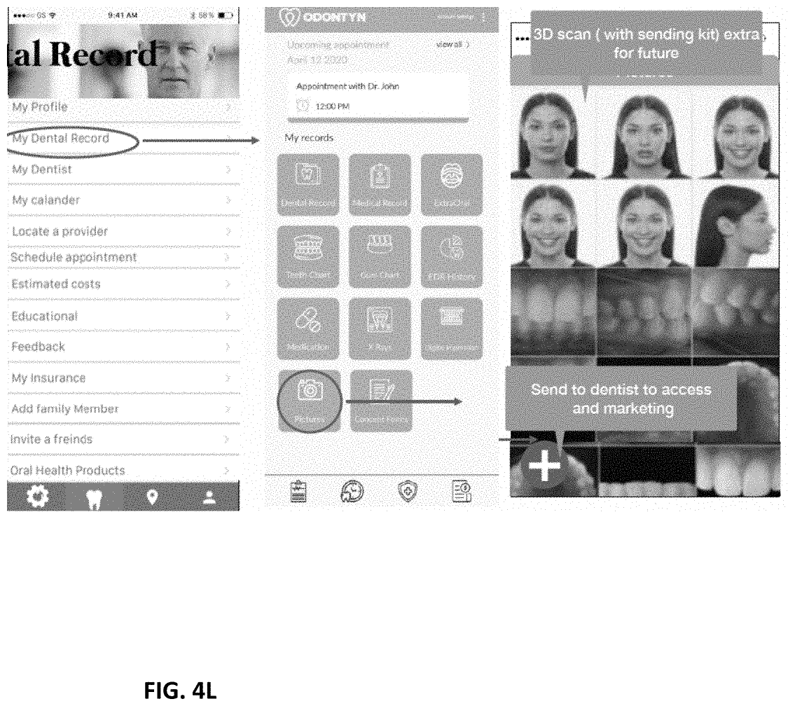

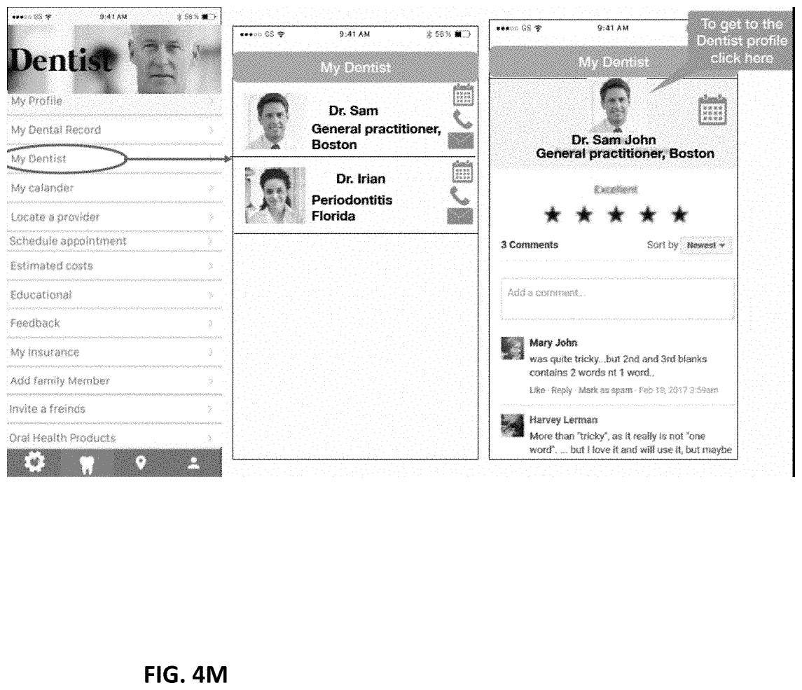

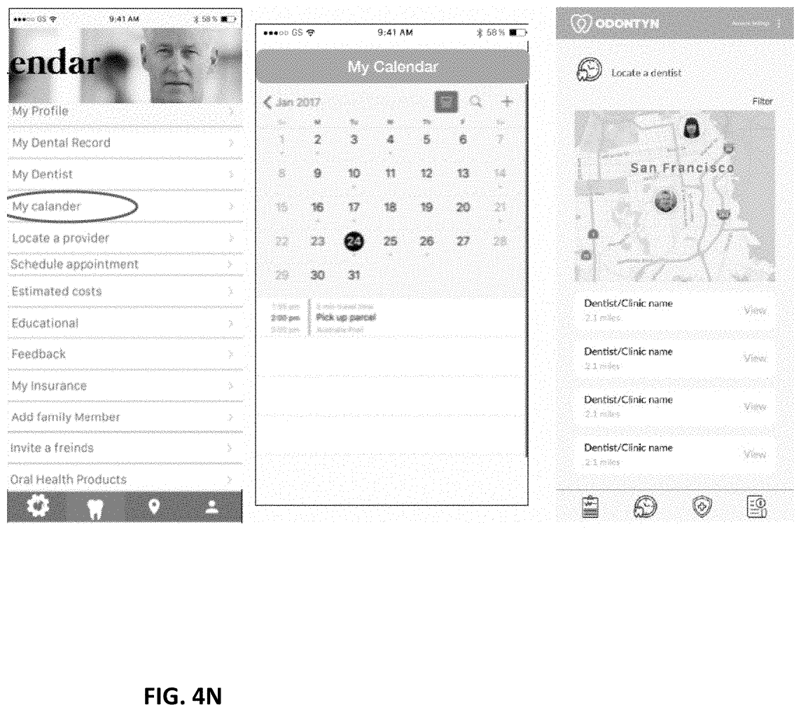

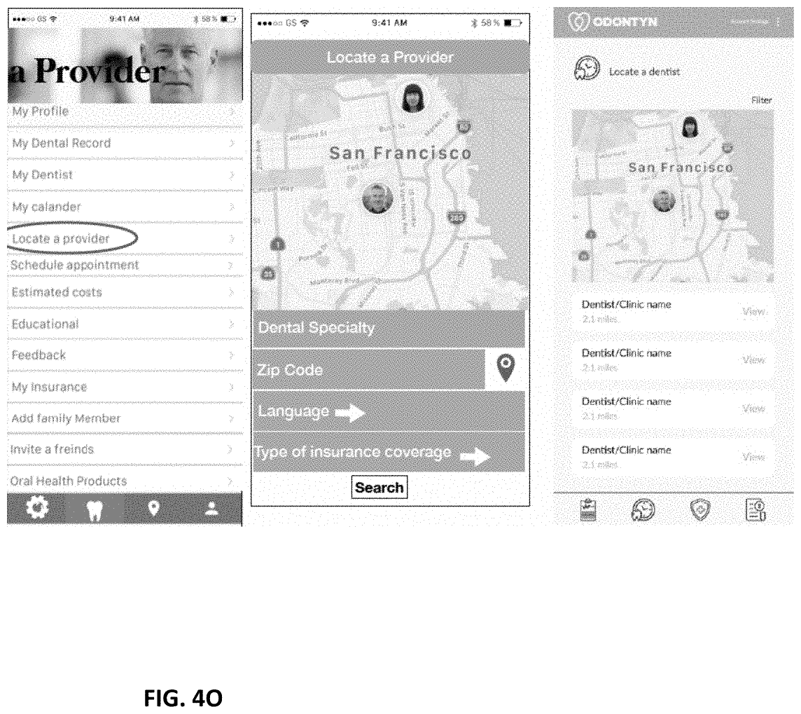

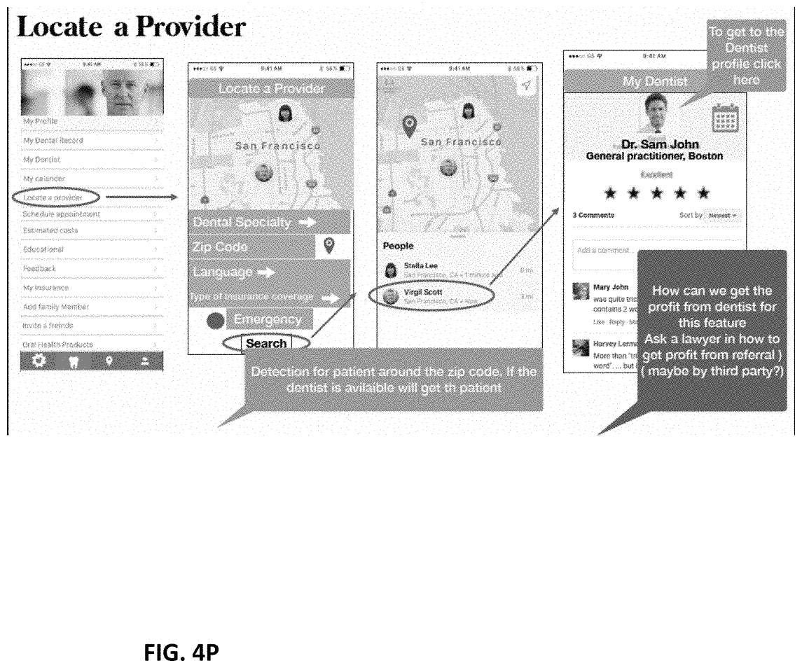

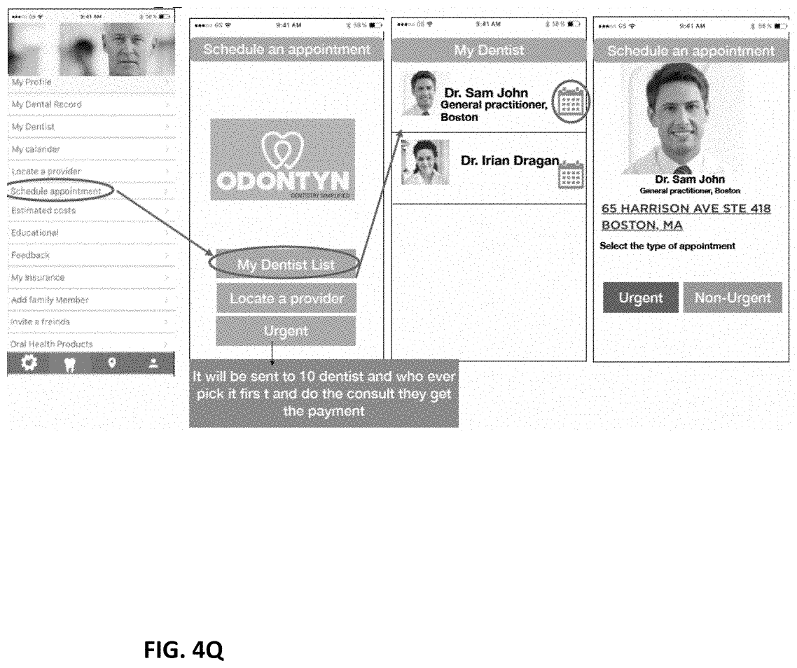

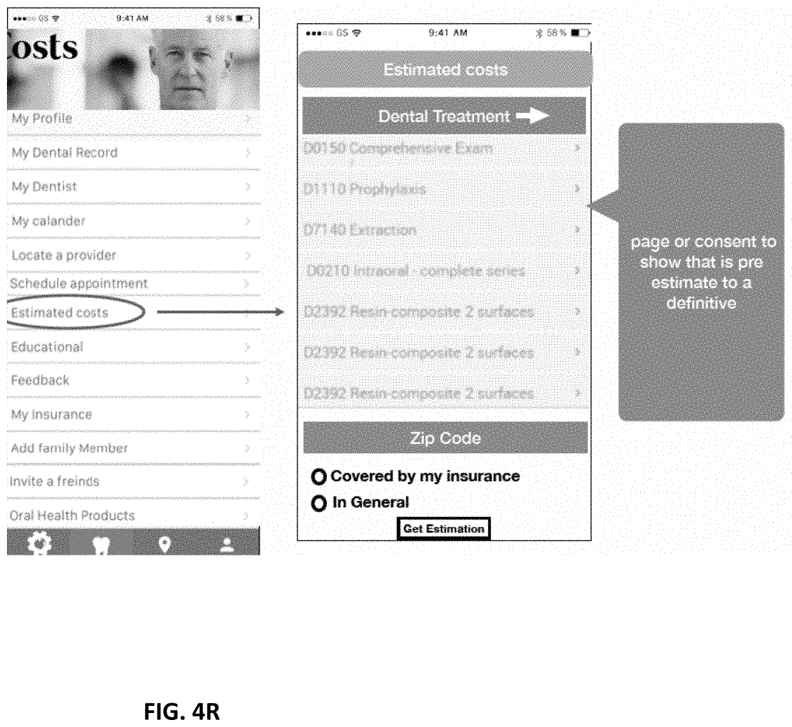

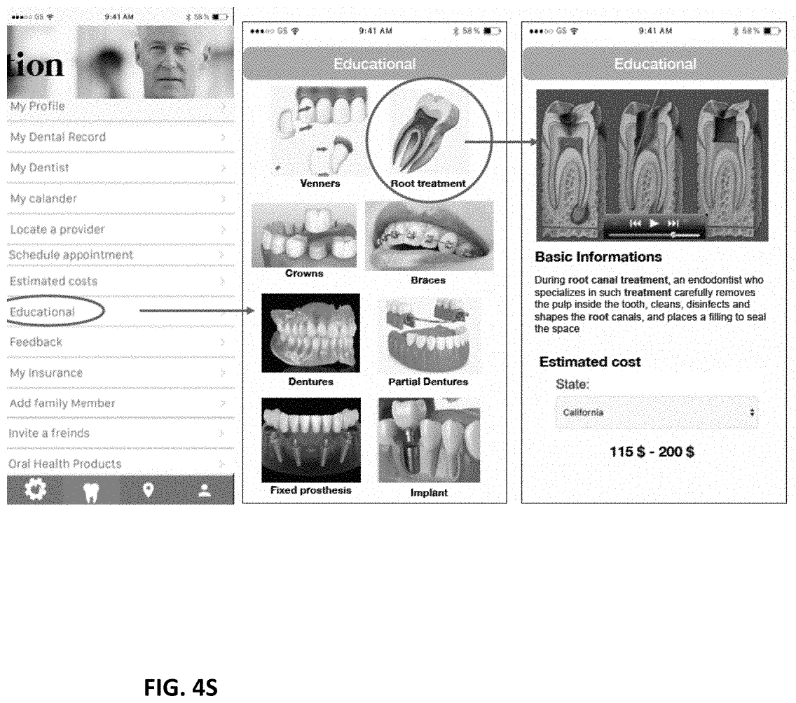

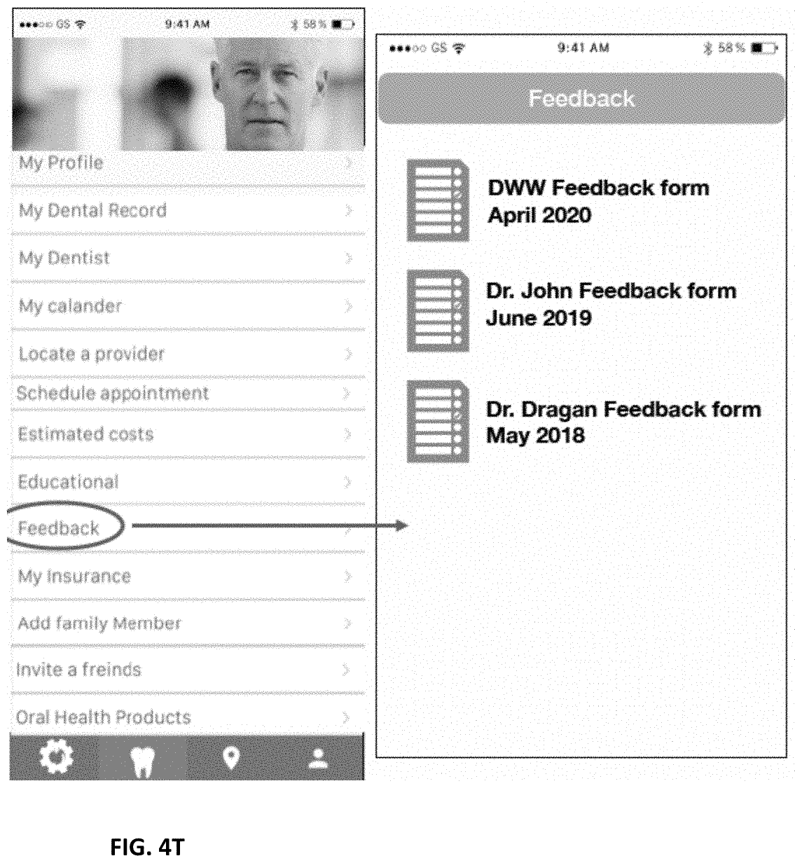

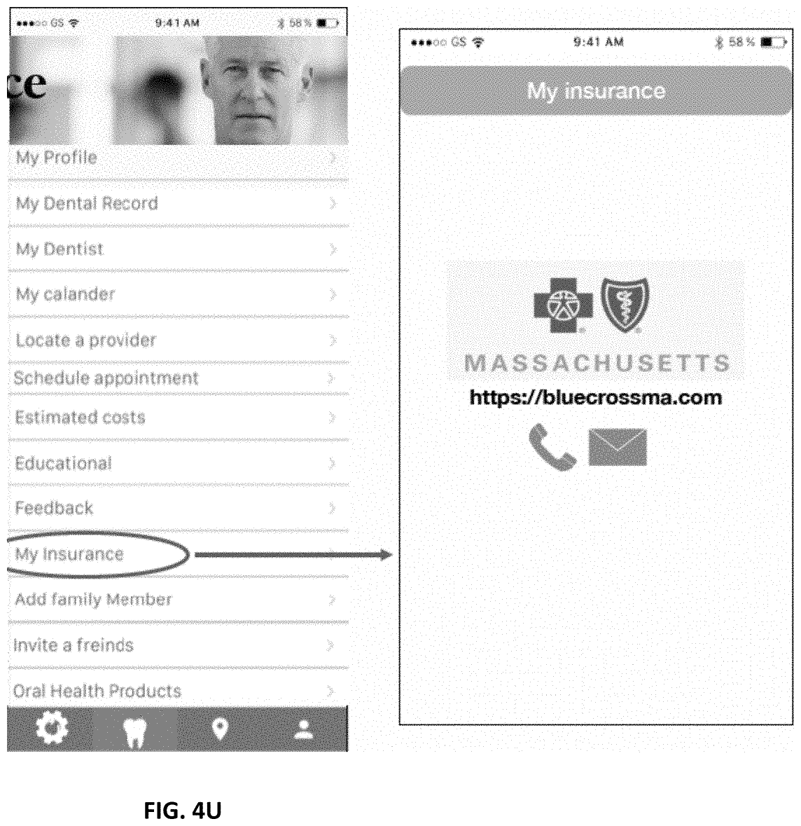

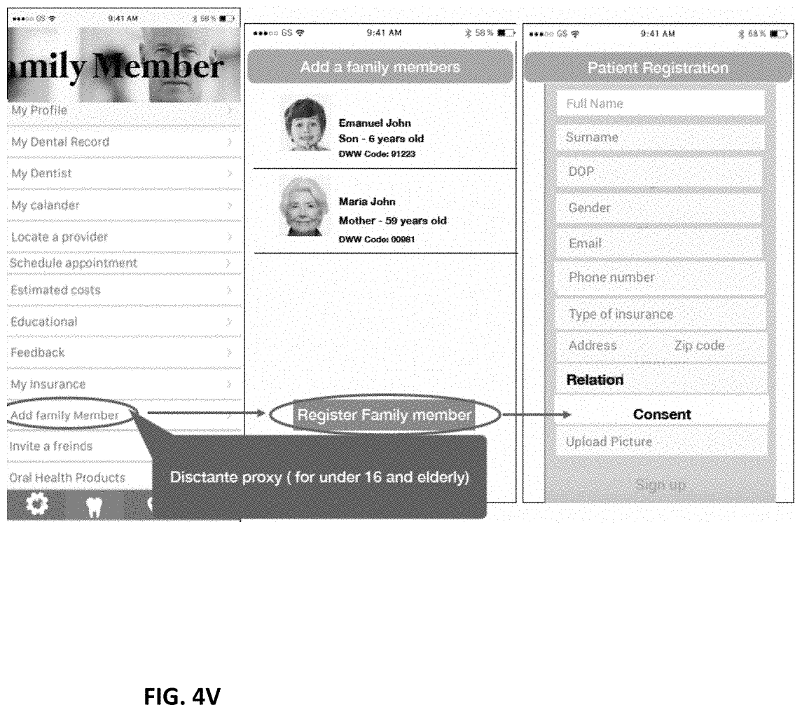

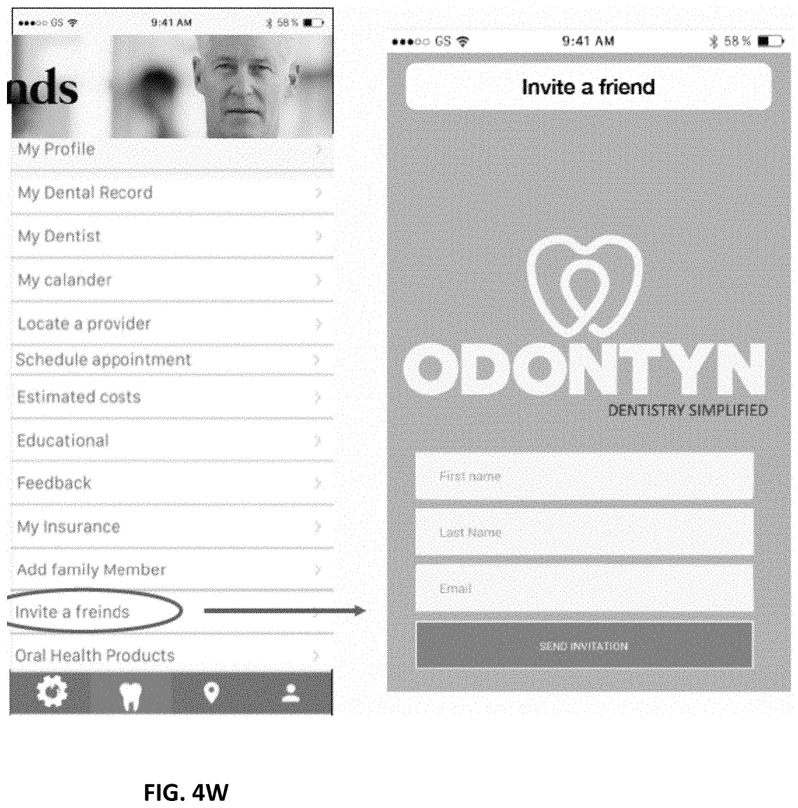

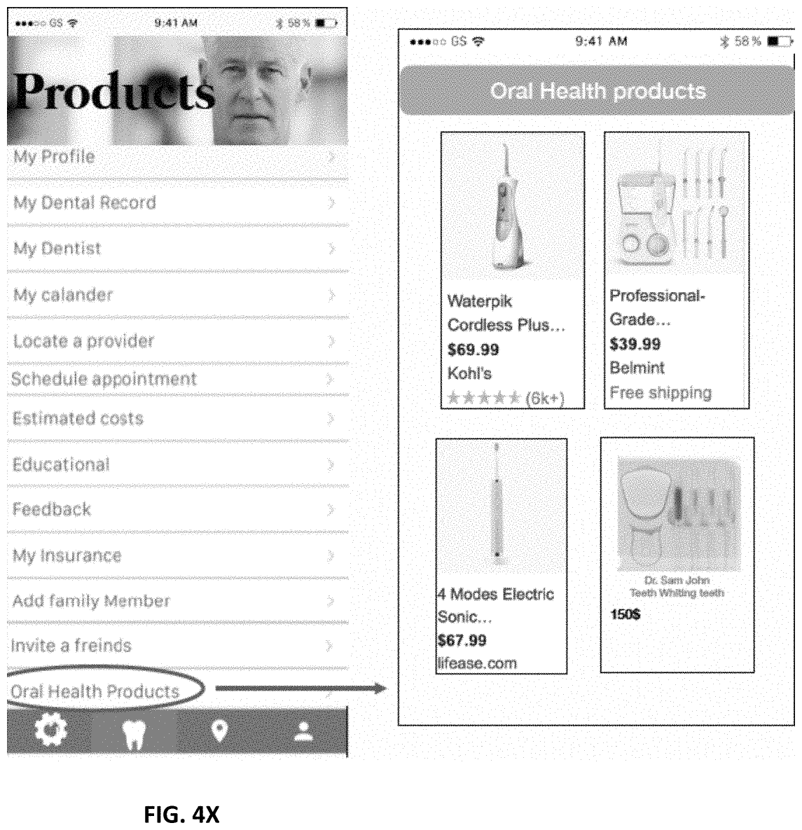

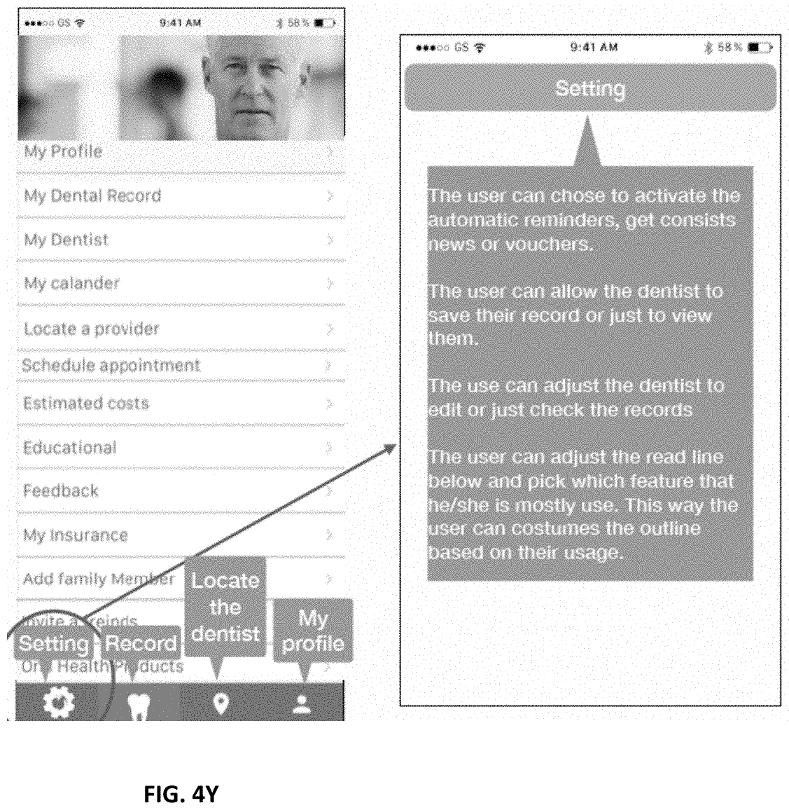

The functionalities of the patient platform can be better understood with reference to FIGS. 4A-4Y. The following is a summary of exemplary functions that the patient platform provides for each user.
My profile: Patient will register with Name, gender, date of birth, phone number, email, password, insurance coverage if exist, zip code, and upload their picture (optional).
My dental record: Upload and integrate EDR to show odontogram and periodontal charts, EDR history, appointment history (showing appointment date, time, procedure and provider name), and radiographic and digital impressions.
Schedule appointment: Tele dentistry (consultations) and integrated in-clinic appointments.
Locate a provider: GIS dentist map, filter by specialty, language, geographic and insurance coverage.
Calendar integration of appointments with reminders and dental hygiene motivational options available.
Estimate costs: Average cost per cities and their insurance coverage.
Educational: Dental education procedures/videos can be viewed by the patient and shared from the dentist.
Royalty/Discount Program: Discounts negotiated with dentists for patient loyalty to their clinic.
Feedback: a survey with set questions from the app, reviews, and ratings.
My insurance: view personalized insurance provider information including rate of savings and money left in plan.
Designate a health proxy that may access your health information and aid in your appointment scheduling.
Invite a friend by adding their name and either phone number or email.

A more detailed description of each function of the patient platform is summarized in Table I below.

TABLE I

| | | API functional |
|---|---|---|
| Feature | Sub-category | specification |
| My profile | Name, gender, date | When the user register, |
| | of birth, phone | specific important feature |
| | number, email, | should be included: first |
| | password, confirm | name, surname, DOB, active |
| | password, insurance | mobile number, email |
| | coverage if exist, | address, dental insurance |
| | address, zip code, | with the type of packages (is |
| | upload picture | exists), address (street |
| | (optional). | number, APT/suite, city, |
| | | state, country, zip code), |
| | | password, confirm password, |
| | | upload picture (optional). |
| | Create patient | Once the patient registered, |
| | code number | an automatic patient code |
| | | number should be provided |
| | | for confidentiality. This code |
| | | will be mainly used to |
| | | communicate with other |
| | | professions. |
| | Forgot password | The ability of the user to |
| | | change the password in |
| | | signing in, by requesting |
| | | forgot password, then |
| | | automatic email verification, |
| | | then check his email to |
| | | confirm. Change the |
| | | password afterwards. |
| | Change password | Once the user register, in |
| | | “My Profile” icon, patient |
| | | able to change the password, |
| | | after mobile verification |
| | | “OPT” |
| | Collect points | Once the user, identified by |
| | (loyalty | his code, schedule 5 |
| | program) | apportionments, write 3 |
| | | comments and rate the |
| | | dentists. The user will get a |
| | | point, after reaching out to |
| | | 10 points, the user will get a |
| | | voucher from the app and |
| | | reward by 5 to 10% discount |
| | | by the coming appointment. |
| | | In specific procedures (ex: |
| | | whiting, cleaning, restoration |
| | | (filling))) |
| | | The ability of the users to |
| | | check their collected points. |
| | Offers and voucher | the user will get a voucher |
| | and discounts | from the app and reward by |
| | | 5 to 10% discount by the |
| | | coming appointment. In |
| | | specific procedures (ex: |
| | | whiting, cleaning, restoration |
| | | (filling)) |
| | | An automatic message of the |
| | | offers/discounts and updated |
| | | (newsletter) from the clinic |
| | | or the dentists that the patient |
| | | contacted. |
| | Motivations messages | Once the user register, the |
| | | motivation messages is |
| | | automatically activated, |
| | | unless the user un-activate it |
| | | manually. |
| | Automatic: Reminder | Automatic: Reminder |
| | motivations messages | motivations messages every |
| | (for example: hygiene | night of brushing. |
| | and positive messages | Also, weekly reminder of the |
| | (smile)) | importance of oral hygiene |
| | | in relation to (oral health, |
| | | social, & systematic health) |
| | | this will be an educational |
| | | tool to not neglect oral |
| | | health. |
| | | In addition, a weekends |
| | | positive messages in regard |
| | | to happy healthy smile, |
| | | positive & bright weekend. |
| My Dental | Upload EDR (Electronic | The ability of the user to |
| Record | Dental record) | upload images (by using “+” |
| | | in the right end of the screen) |
| | | of previous dental EHR. |
| | | Specially from their old |
| | | dentists who does not use an |
| | | EHR software that digitally |
| | | transferred and recognized |
| | | by ODONTYN software/or |
| | | from old dentist who still use |
| | | manual dental records. |
| | | Alternative option is to have |
| | | Scanner functionality |
| | | “Natural language |
| | | processing” can be used to |
| | | unify and extract the main |
| | | brief data on the previous |
| | | dental procedure and show |
| | | them in one schedule that |
| | | easy to the patient and |
| | | dentist to navigate. Important |
| | | information in; date of |
| | | procedure, tooth or region, |
| | | procedure code, and |
| | | diagnosis code. |
| | | The user will be able to see |
| | | brief history of the produce |
| | | code, tooth number and date. |
| | | Color coding to show the |
| | | stage of the procedure |
| | | (“green” treatment |
| | | competed, “yellow” |
| | | treatment on hold and “red” |
| | | treatment is planned) |
| | My Teeth chart | Provide 32 teeth templet. |
| | | The ability of the patient to |
| | | understand the (missing |
| | | tooth, decayed tooth, |
| | | restored tooth with the |
| | | selection of the materials, |
| | | root treated tooth, crowns |
| | | tooth. . .). |
| | | Brief schedule of the date, |
| | | teeth number, surface, |
| | | description and provider |
| | | code in editing or adding in |
| | | “my teeth chart”. |
| | | The patient is not allowed to |
| | | add or edit the form |
| | | Voice record with auto type |
| | | will be implemented |
| | Gum chart | Periodontal table, that shows |
| | | the user periodontal |
| | | examination by either |
| | | dentist, periodontist or |
| | | hygienist. Examinations and |
| | | number that includes (pocket |
| | | depth, recession, calculus, |
| | | clinical attachment loss). |
| | | A scale of numbers that |
| | | shows the current |
| | | periodontal condition of the |
| | | patient: if the numbers from |
| | | 1 to 3 (appear green), if the |
| | | number 4-5 appear yellow, if |
| | | the numbers 6 and above red. |
| | | An automatic the number |
| | | colure scale will educate the |
| | | patient with easy way to |
| | | identify the areas that need to |
| | | maintain and follow up. |
| | | Brief schedule of the date, |
| | | periodontal examination and |
| | | provider code in editing or |
| | | adding in “my gum chart”. |
| | | The patient is not allowed to |
| | | add or edit the form. |
| | | Voice record with auto type |
| | | will be implemented. |
| | Extra oral | Soft tissue (Intra oral and |
| | examination | extra oral) date, description |
| | | and provider noted |
| | | Skeletal classification (class |
| | | 1, 2, 3) options, so the dentist |
| | | can select one. |
| | | cross bit; dentist can select |
| | | form 2 options anterior or |
| | | posterior, right or left. |
| | | Overjet; with 3 options |
| | | (mild, moderate, sever) |
| | | Overbite; with 3 options |
| | | (mild, moderate, sever) |
| | | TMJ symptoms description |
| | | by dentist. |
| | | Parafunctional habits |
| | | description by the dentist. |
| | | The patient is not allowed to |
| | | add or edit the form. |
| | EDR History | History of procedure that |
| | | carried out; the procedure |
| | | code, the tooth number, the |
| | | date, & dentist code. |
| | | The user once clicks on the |
| | | procedure day a brief |
| | | description of the dental |
| | | procedure or consultation |
| | | (wither emergency or 2^{nd} |
| | | opinion) carried out that |
| | | been written by the dentist |
| | | (in dentist version). |
| | | Color coding to show the |
| | | stage of the procedure (green |
| | | competed, yellow on hold |
| | | and red is planned) |
| | EMR (Electronic | Medical conditions: (which |
| | medical record) | the dentist can update only) |
| | | Vital signs with BMI - this |
| | | can be linked to the apple |
| | | watch in the user is using. |
| | | Medical condition the edited |
| | | by either the patient or |
| | | dentist with date diagnosed |
| | | and dentist code if been |
| | | edited by him. |
| | | Color coding to show the |
| | | stage of the procedure (green |
| | | controlled, yellow active and |
| | | red uncontrolled) |
| | My Appointments | History of both Upcoming |
| | | and previous appointments. |
| | | That shows the type (tele or |
| | | in clinic), the date and time |
| | | and provider name or code. |
| | X-rays | The user can upload their |
| | | own radiograph (in both |
| | | DICOM and JPG) |
| | | The user can access to their |
| | | own radiograph that the |
| | | dentist uploaded. |
| | | An extra feature in making |
| | | marks upon sending to |
| | | further explain to the |
| | | provider, but the marks |
| | | won't be saved into the |
| | | original radiograph image. |
| | | The user can share or send |
| | | the radiograph to the app |
| | | text, email and mobile |
| | | device). |
| | | DICOM file the user should |
| | | pay for the extra feature, as |
| | | this will require extra |
| | | storage. |
| | Digital impression | This is an upgrade. If the |
| | | user had an oral digital |
| | | scanning, they can upload |
| | | themselves (STL. file) or the |
| | | dentist uploaded for them. |
| | | The user can access to their |
| | | STL file. |
| | | The user can share and send |
| | | the digital file. |
| | | STL file the user should pay |
| | | for the extra feature, as this |
| | | will require extra storage. |
| | Pictures | Front view of the patient in |
| | | relaxed |
| | | Frontal view of the patient |
| | | smiles socially |
| | | Frontal view smile maximum |
| | | Side view of the patient |
| | | relaxed |
| | | Side view of patient smile. |
| | | Close up smile photo |
| | | Extraoral picture retract lips |
| | | foil profile in MIP |
| | | Extraoral picture retract lips |
| | | foil profile in slight opening |
| | | Extraoral picture Retract lip |
| | | upper teeth (with back |
| | | separation) |
| | | Intra oral retracting in MIP, |
| | | protrusion, lateral excursion. |
| | | Intra oral picture occlusal |
| | | upper |
| | | Intra oral picture occlusal |
| | | Lower |
| | Medications | It will be divided to three |
| | | sections; currently taken, |
| | | recently prescribed, and |
| | | allergies). |
| | | Color coding system to ease |
| | | for the patient and dentist to |
| | | overview; green in need, |
| | | yellow temporarily |
| | | Once the dentist prescribes, |
| | | and red stop taking) |
| | | The mediation to the patient |
| | | stating (the name of |
| | | mediation, dosage, duration |
| | | and instruction). The user |
| | | will get an automatic |
| | | reminder of the prescribed |
| | | medication align to their |
| | | schedule. |
| | Billing | The patient will keep track of |
| | | their billing, insurance or |
| | | clinic refines and payments. |
| | | It will include foe date of |
| | | procedure, the treatment |
| | | code and foe dental clinic |
| | | location. |
| | Consent forms | Patient will receive a consent |
| | | form from the dentist prior to |
| | | procedures or signing |
| | | treatment plan. Patient will |
| | | be able to keep and store |
| | | their consent forms in the |
| | | app. |
| | | Patient can sign consent |
| | | form also prior of through |
| | | tele schedule appointment or |
| | | consultations. |
| My Dentist List | | A list of dentists that been |
| | | seen in clinic or scheduled |
| | | by tele dentistry. |
| | | Its' up to the patient to |
| | | decide and save the dentist to |
| | | “My Dentist”. |
| | | the user can click on the |
| | | dentist picture profile or the |
| | | name; where the dentist rate |
| | | and comments appear. If the |
| | | patient would like to know |
| | | more about the dentist, they |
| | | can click on the dentist |
| | | picture to access to dentist's |
| | | profile. |
| | | Icon of schedule, call, or text |
| | | the patient appears in from |
| | | each dentist name in” my |
| | | dentist” page. |
| Schedule | | Three options: from my |
| Appointment | | dentist list, locate a provider, |
| | | urgent. |
| | | If the user selects “my |
| | | dentist list”, the page |
| | | appears, and patient schedule |
| | | the appointment by selecting |
| | | by 2 options either be urgent |
| | | or non-urgent appointment. |
| | | Urgent appear in red and |
| | | non-urgent upper in green. |
| | Urgent | The user will fill a form the |
| | Tele-dentistry | main presenting complains |
| | | “Select the type of |
| | | emergency” and “open |
| | | comment box”. The user |
| | | specifies the type of |
| | | communication texting or |
| | | mobile audio/virtual call. |
| | | Option of the main |
| | | presenting complain will be |
| | | provided and comment box |
| | | to explain further. |
| | | Then the user can filer the |
| | | time to schedule, the type of |
| | | dentists and zip code. A |
| | | selection of dentist will be |
| | | provided, once the use finds |
| | | his match with his/her |
| | | convenient time, they |
| | | schedule the appointment |
| | | directly. |
| | | If the patient selects text, a |
| | | text box appears, where they |
| | | specify and explain their |
| | | situation. |
| | | The “type of emergency and |
| | | “comment automatically |
| | | appear in the chat, once the |
| | | patient select it. |
| | Non-Urgent | Two option of the non- |
| | as a 2^{nd} | urgent appointment; the first |
| | online opinion | option “2^{nd} online opinion, |
| | schedule clinic | where the user fills a form |
| | appointment | with main presenting |
| | | complain with a list and |
| | | comment box for further |
| | | explanation. The user can |
| | | access to their radiograph |
| | | and select the related |
| | | radiograph, and similar to |
| | | their digital scan and dental |
| | | record examination that they |
| | | want to share it. Also, the |
| | | user can upload directly intra |
| | | and extra oral picture from |
| | | the app or browser. The user |
| | | prefers to communicate by |
| | | ticking the following |
| | | options: texting, mobile |
| | | audio call or virtual call. |
| | | Then the user can filer the |
| | | time to schedule, the type of |
| | | dentists and zip code. A |
| | | selection of dentist will be |
| | | provided, once the use finds |
| | | his match with his/her |
| | | convenient time, they |
| | | schedule the appointment |
| | | directly. |
| | | For the 2^{nd} option “schedule |
| | | clinic appointment” the user |
| | | can skip the first part and |
| | | schedule directly with |
| Locate a | | Dentists list with the type of |
| provider | | insurance they accept, |
| | | language they speak, zip |
| | | code and profession. |
| | GIS dentist, filter by | The user can filter the kind |
| | specialty and insurance | of dentist they are searching |
| | coverage | for: list of insurance name |
| | | and packages, language, zip |
| | | code, profession and |
| | | availabilities. |
| | | Once the filter has carried |
| | | out by the user, a map and |
| | | list of dentists will be |
| | | provided, by clicking on the |
| | | dentist, the name and rating |
| | | will appear. The user can |
| | | click in the dentists an access |
| | | his portfolio or page and |
| | | check his account. Once the |
| | | user accepts then the user |
| | | will request to schedule |
| | | appointment either urgent or |
| | | non-urgent . . . the rest similar |
| | | steps to “Schedule |
| | | Appointment” |
| Calendar | their appointments | A user can access directly to |
| | | check their appointment |
| | | schedule and synergize it |
| | | with their mobile or google |
| | | calendar. |
| Estimated costs | Average cost per | This feature will provide the |
| | cities and their | estimate treatment code per |
| | insurance coverage | zip code or a state. |
| | | An additional feature will be |
| | | provided in the future, once |
| | | the user specifies the type of |
| | | insurance package and the |
| | | discounts. The user will add |
| | | the dentist cost and the |
| | | insure automatically provide |
| | | an estimate of how much |
| | | they will refund them. |
| Educational | Dental education | A selection of dental |
| | procedures/videos | treatment plan will be |
| | | provided with visual brief |
| | | description for each |
| | | procedure. |
| | | Also, a search engine that |
| | | access them to PubMed and |
| | | ADA journals. |
| Feedback | survey with set | Three sections: the 1^{st} section |
| | questions from the | is to fill out a feedback form |
| | app, reviews and | for both using the app and |
| | rating to the | about the provider after |
| | dentists. | checking out. The form will |
| | | be provided later. |
| | | the 2^{nd} & 3^{rd} section to rate |
| | | and leaves a comment to the |
| | | provider. |
| My insurance | communicate directly | Direct sent an email to the |
| | with the insurance. | insurance, that the patient |
| | | registered in their profile. |
| Oral health | upgraded version | (Future upgrade) |
| products | | Users can order and rate oral |
| | | health products; ex: |
| | | electronic tooth brush, water |
| | | pik . . . etc |
| | | This feature will be provided |
| | | once dental companies |
| | | partner up with us. |
| Register a | Maximum of 4 members | Option to register and have |
| family member | (caregiver) | all the features for the family |
| | | members |
| Invite a | Friend name, email, | A way to inform their family |
| friend | or phone number | and friends about the app. |
| | | Once the user invites a friend |
| | | and their friend registered, |
| | | the sender will get 1 reward |
| | | point. |

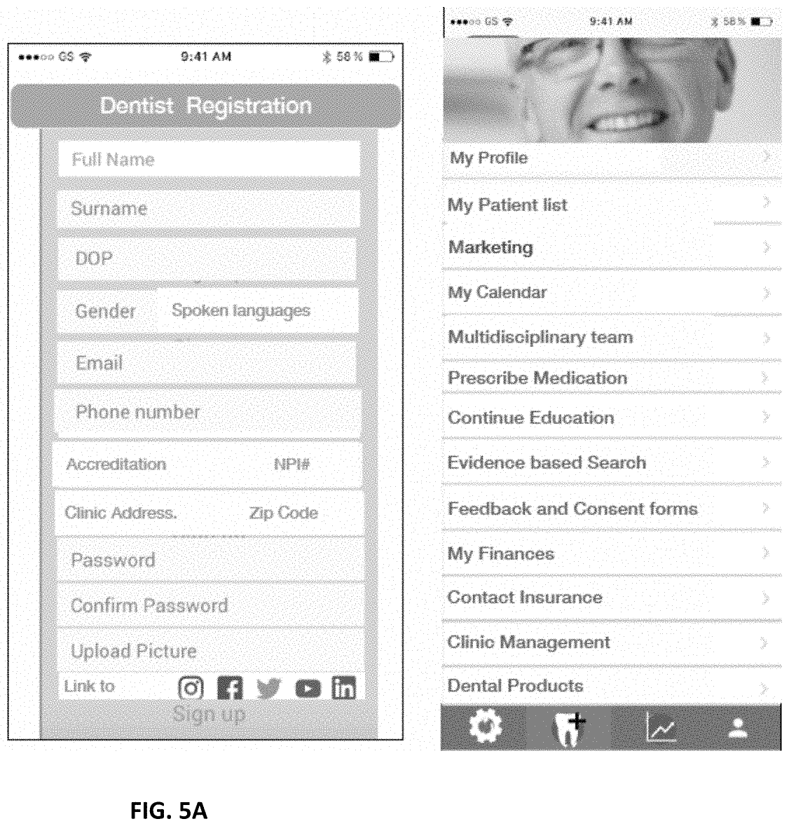

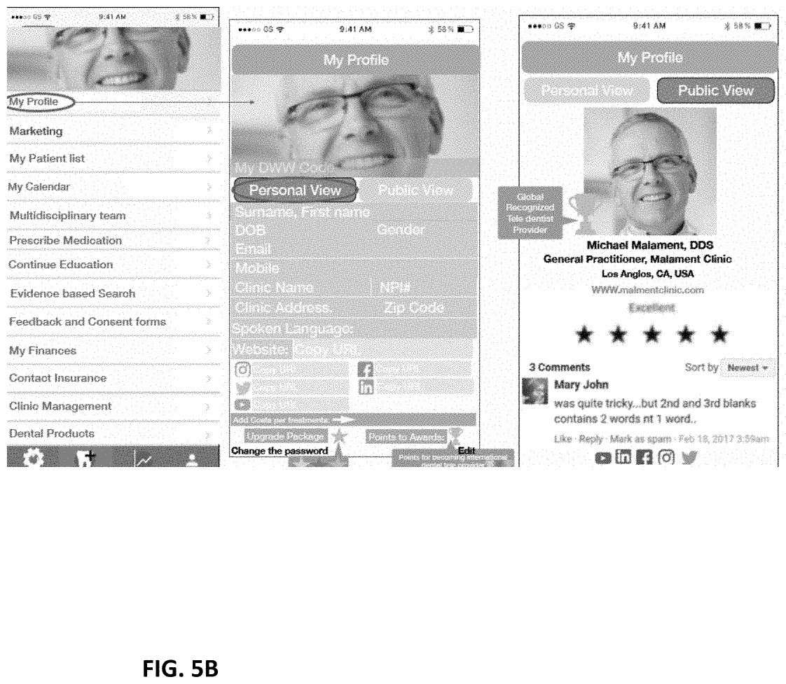

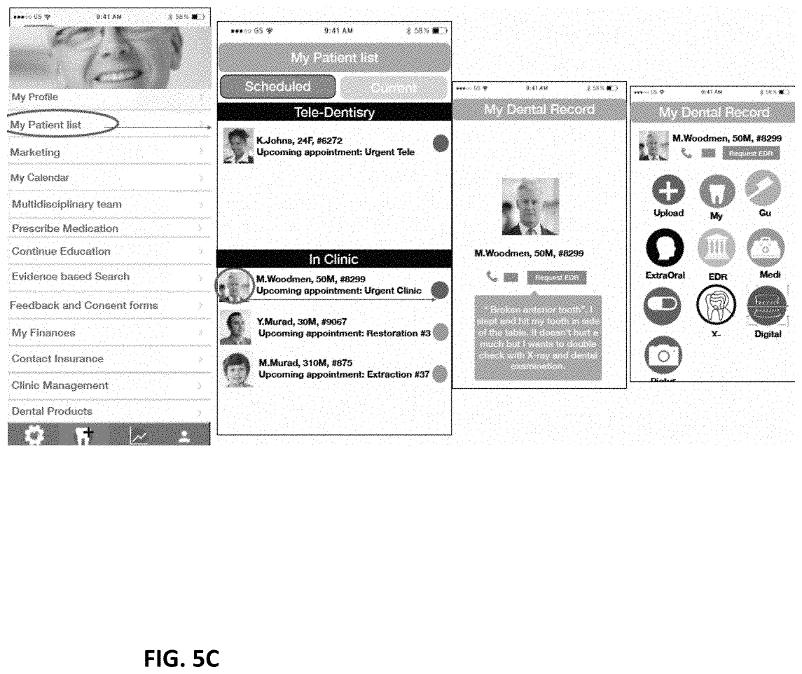

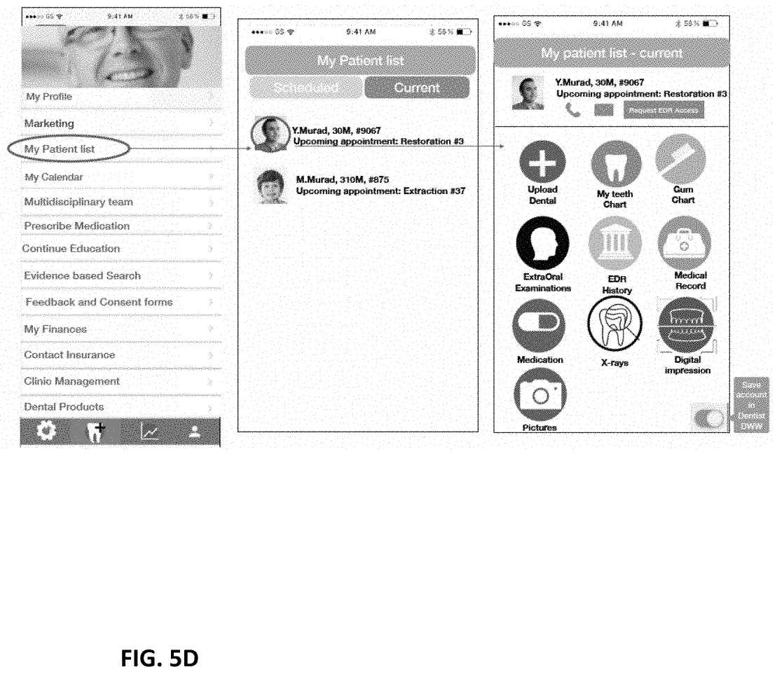

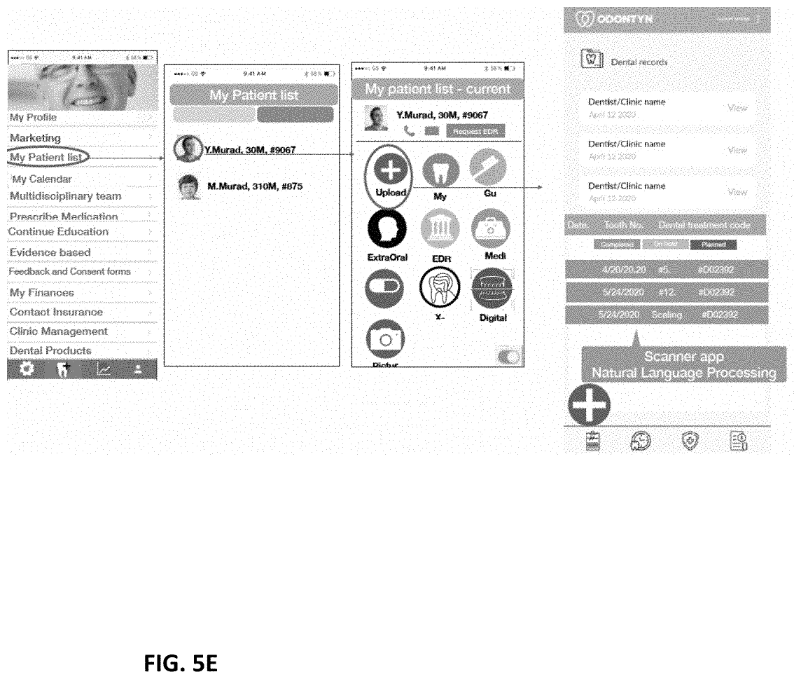

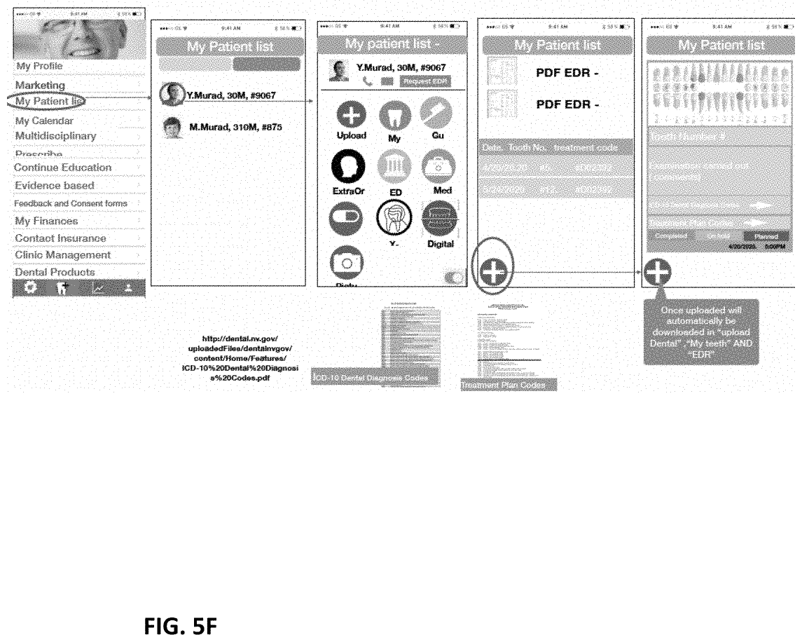

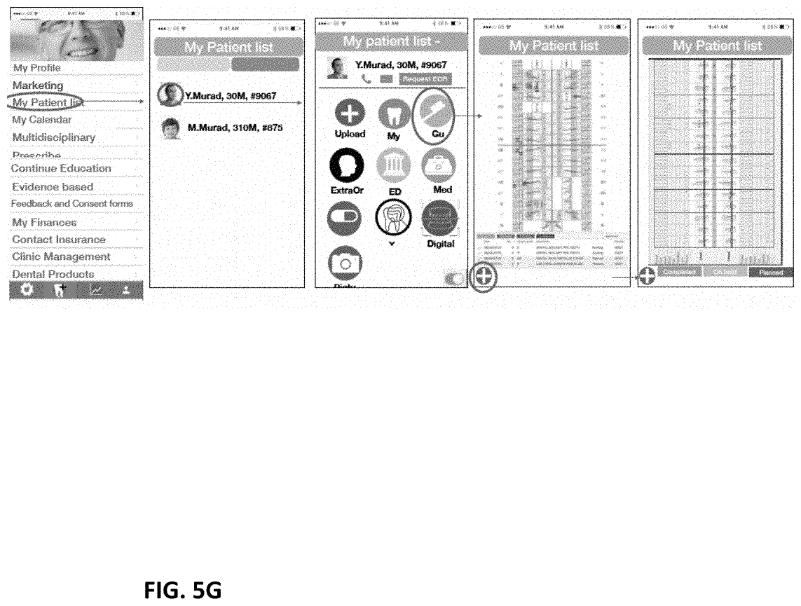

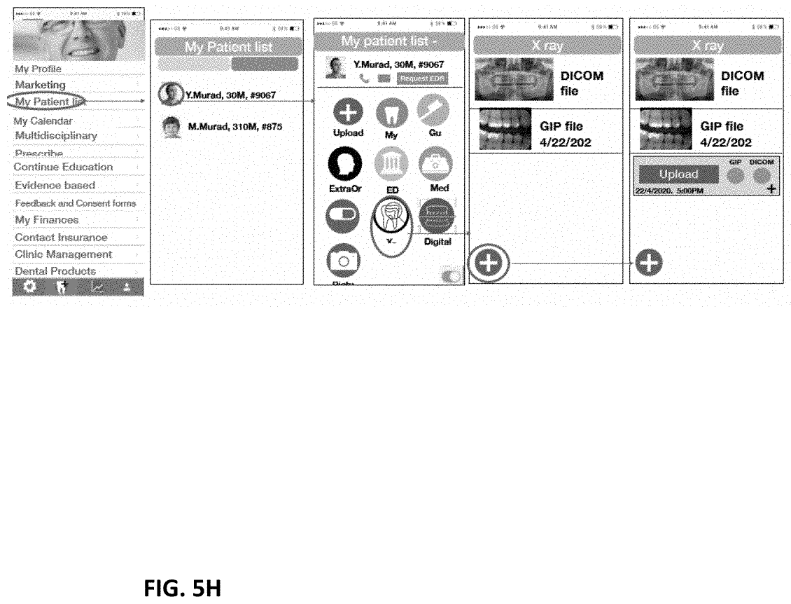

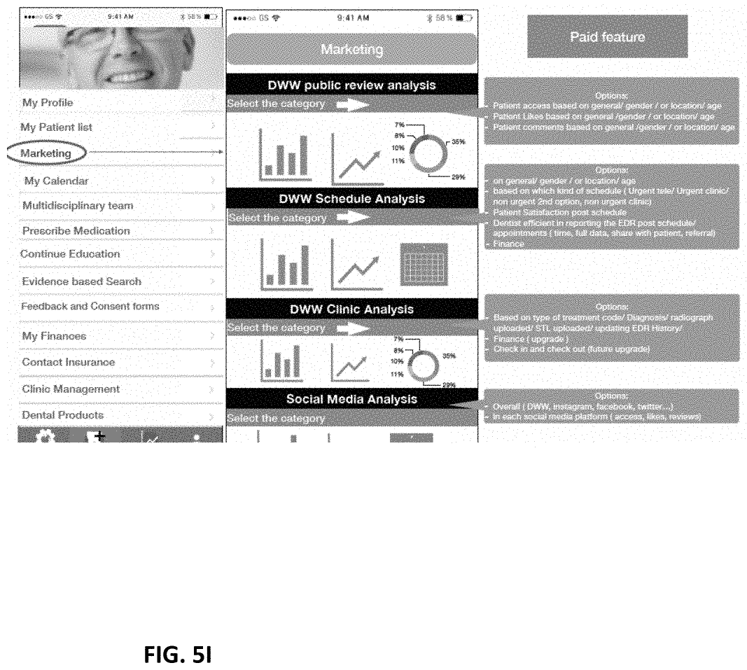

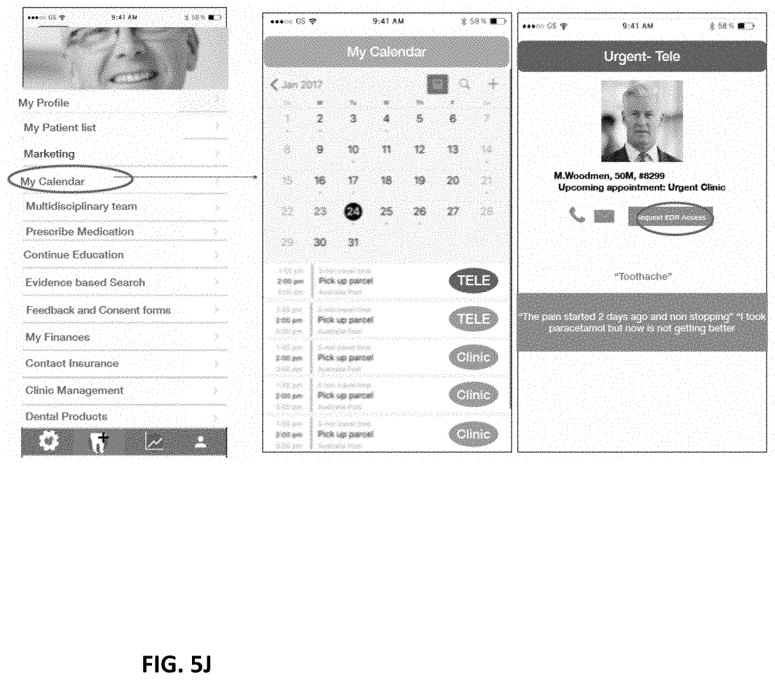

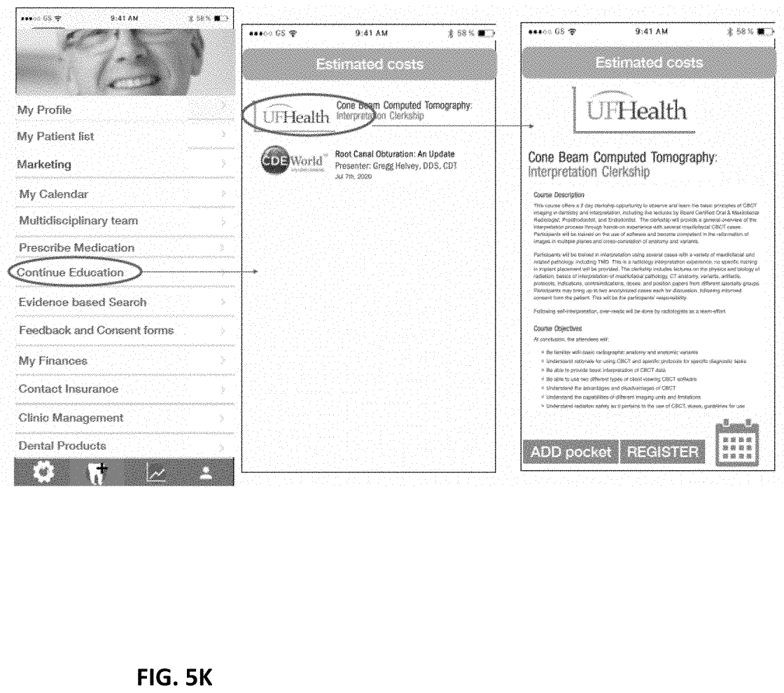

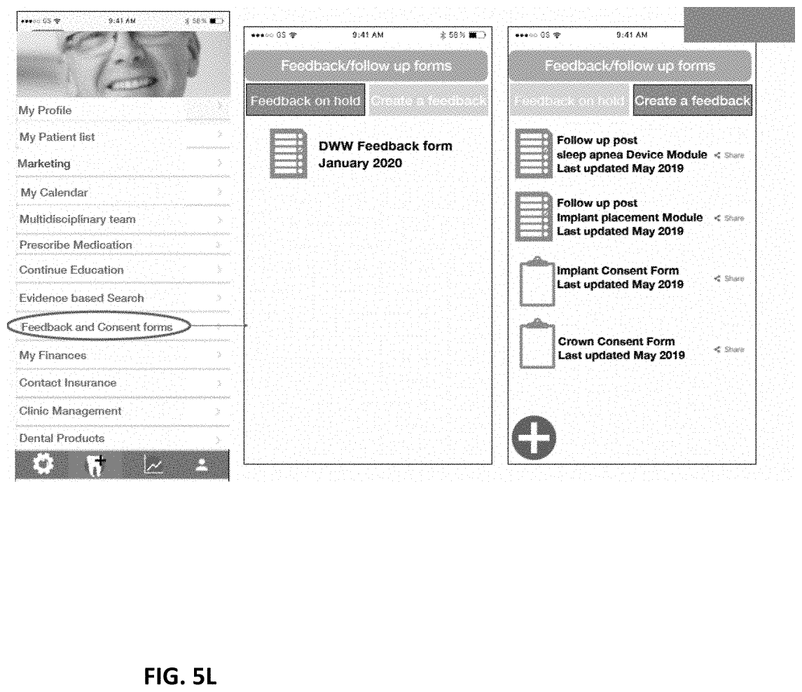

The functionalities of the dentist platform can be better understood with reference to FIGS. 5A-5L. The following is a summary of exemplary functions that the dentist platform provides for each user.
My profile: Dentist will register with name, accreditation, specialty, gender, date of birth, zip code, name and location of the clinic, spoken language, upload picture, link to social media)-estimated cost per treatment (optional).
Marketing: Reviews/ratings and linking to other social media accounts—analysis of comments, demographics, surveys, likes, ratings.
My patient's list: Scheduled Patients: tele, urgent, non-urgent designations with name, gender, DOB, presenting complain. Current Patients: Patient name list, treatment code, diagnosis, age, gender, tele or 2nd opinion or in the clinic. Once patient's row is clicked, container box with: Name/Gender/DOB/Zip code and app code's, history of Dental clinical notes, diagnosis, treatment code—contact made by tele or 2nd opinion or in a clinic. Dentist will have ability to contact patients by text or phone and have immediate access to their EHR. Once the dentist access to the patient's record; the dentist will allow to edit, upload and update the patient record through the website. By implementing the natural processing language AI, the uploaded EDR file will be organized and illustrated in the area that it should be (ex; dental and periodontal chart, intraoral pictures, x-ray, record (presenting complain/examination findings/diagnosis/treatment code recommended).
Multidisciplinary team: to refer and keep track of referred patients.
Prescribe medication: direct to patient's zip code closest pharmacy—potential integrate with another pharmaceutical application.
Feedback and consent: allows dentists to manage consent forms and feedback received from patients.
Contact insurance: communicate directly with the insurance (send pre-estimation and claims)
Clinic management: (check-in/check-out. thank you for a visit) (software dental records).
My finances: allows dentists to manage finances.
Continued education: allows dentist to find registered courses/workshops/webinars.
Dental products: provides information and ability to purchase dental products as well as to recommend to patients.
Evidenced-based and Research: provides resources for conducting research.

A more detailed description of each function of the dentist platform is summarized in Table II below.

TABLE II

| | | API functional |
|---|---|---|
| Feature | Sub-category | specification |
| My profile | Dentist “My profile” | |
| | divided into two sections | |
| | “personal view” and | |
| | “Public view”. | |
| | In the personal view: | A specific requirement that |
| | Name, accreditation, | the dentist have to provide |
| | specialty, gender, date | in registration: first name, |
| | of birth, zip code, name | surname, NPI, accreditation, |
| | and location of the clinic, | the name of the current clinic |
| | spoken language, upload | practice, the current clinic |
| | picture, link to social | address: (street number, |
| | media. | suite/floor, city, state, |
| | | country, zip code), spoken |
| | | language, upload |
| | | picture(optional), URL of the |
| | | website (optional), link to |
| | | social media (Instagram, |
| | | Facebook, LinkedIn, |
| | | YouTube). |
| | Create dentist | Once the dentist registered, |
| | code number | an automatic patient code |
| | “My ODONTYN | number should be provided |
| | Code” | for confidentiality. This code |
| | | will be mainly used to |
| | | communicate with other |
| | | professions. |
| | Forgot password | The ability of the user to |
| | | change the password in |
| | | signing in, by requesting |
| | | forgot password, then an |
| | | automatic email verification, |
| | | then check his email to |
| | | confirm. Change the |
| | | password afterwards. |
| | Change password | Once the user register, in |
| | | “My Profile” icon, patient |
| | | able to change the password, |
| | | after mobile verification |
| | | “OPT” |
| | “Points to IDP | “IDP Intentional Dental |
| | Awards” | Provider Awards” |
| | | Once the dentist reaches to a |
| | | specific rate and |
| | | requirements, the dentist will |
| | | receive an award of |
| | | becoming an internal dental |
| | | provider award (Bronze, |
| | | silver, gold and Dimond). |
| | | Bronze Award; dentist |
| | | should have 50 sustained |
| | | patients from ODONTYN |
| | | platform, performed at least |
| | | 30 tele dentistry, 50 updated |
| | | reports EDR in patient |
| | | portals in any form; tooth |
| | | chart, gum chart, history, |
| | | upload images/radiographs. |
| | | Receive at least 40 rates, 3.5 |
| | | out 5 stars and 20 comments. |
| | | Silver Award; dentist |
| | | should have 200 sustained |
| | | patients from ODONTYN |
| | | platform, performed at least |
| | | 50 tele dentistry, served |
| | | patient in 2 different states, |
| | | 200 updated reports EDR in |
| | | patient portals in any form; |
| | | tooth chart, gum chart, |
| | | history, upload |
| | | images/radiographs. Receive |
| | | at least 100 rates, 4 out 5 |
| | | stars and 50 comments. |
| | | Gold Award; dentist should |
| | | have 500 sustained patients |
| | | from ODONTYN platform, |
| | | performed at least 300 tele |
| | | dentistry, served patient in 3 |
| | | different states, 500 updated |
| | | reports EDR in patient |
| | | portals in any form; tooth |
| | | chart, gum chart, history, |
| | | upload images/radiographs. |
| | | Receive at least 300 rates, |
| | | 4.5 out 5 stars and 150 |
| | | comments. |
| | | Diamond Award; dentist |
| | | should have 1000 sustained |
| | | patients from ODONTYN |
| | | platform, served patient in 5 |
| | | different states, performed at |
| | | least 700 tele dentistry, 1000 |
| | | updated reports EDR in |
| | | patient portals in any form; |
| | | tooth chart, gum chart, |
| | | history, upload |
| | | images/radiographs. Receive |
| | | at least 600 rates, 4.9 out 5 |
| | | stars and 300 comments. |
| | Upgrade Package | Upgrade Package: |
| | | Three different type of |
| | | packages with different |
| | | features (Bronze, Silver, |
| | | Gold). |
| | | Free use: |
| | | Profile in ODONTYN |
| | | 30 patient's storage. |
| | | My calendar |
| | | Tele dentistry and schedule |
| | | patients. |
| | | Continue education. |
| | | Use EVD search and save 5 |
| | | articles in the libarary. |
| | | Bronze Package: 50 patient |
| | | storage, |
| | | Sell dental products |
| | | 1 post marketing. |
| | | My calendar |
| | | Tele dentistry and schedule |
| | | patients. |
| | | Continue education. |
| | | Use EVD search and save 10 |
| | | articles in the library. |
| | | Multidisciplinary referral |
| | | and tracking patient system. |
| | | Package marketing analysis. |
| | | Silver Package: 300 patient |
| | | storage, |
| | | Sell dental products |
| | | 2 post marketing a month. |
| | | My calendar |
| | | Tele dentistry and schedule |
| | | patients. |
| | | Continue education. |
| | | Use EVD search and save 20 |
| | | articles in the library. |
| | | Multidisciplinary referral |
| | | and tracking patient system. |
| | | Package marketing analysis. |
| | | Ready consent forms and |
| | | feedback forms. |
| | | Gold Package: 500 patient |
| | | storage, |
| | | Additional 50$ for 50 |
| | | patients additional. |
| | | Sell dental products |
| | | 3 post marketing a month. |
| | | My calendar |
| | | Tele dentistry and schedule |
| | | patients. |
| | | Continue education. |
| | | Use EVD search and save 50 |
| | | articles in the library. |
| | | Multidisciplinary referral |
| | | and tracking patient system. |
| | | Package marketing analysis. |
| | | Ready consent forms and |
| | | feedback forms. |
| | | My finance features. |
| | | Clinic management feature. |
| | Estimated cost | The dentist can provide the |
| | per treatment | cost of each treatment code |
| | (optional) | in the clinic. A feature will |
| | | help the patient to get an |
| | | estimate amount of the |
| | | budget that need to save |
| | | prior to the appointment. |
| | In Public View: | Dentist profile image, name, |
| | My Rating | specialty, location and |
| | | website. |
| | | The type of IDPA that the |
| | | dentist got. |
| | | The dentist will be able to |
| | | check his rates, and his |
| | | patient reviews and |
| | | comments. |
| | | Link to all the social medial |
| | | profiles. |
| | | Two sections; scheduled |
| | | (tele or clinic) and cunent |
| | Scheduled: | Two sections (tele and |
| | Tele (urgent or | clinic): |
| | 2^{nd} | In Tele-dentistry; patient |
| | opinion) | picture, name, age, gender, |
| | | ODONTYN code, the type |
| | | of schedule tele appointment; |
| | | red is urgent, and green is |
| | | not urgent (2^{nd} opinion). |
| | | The dentist can click on the |
| | | patient image and check the |
| | | “presentation complains and |
| | | any comments from the |
| | | patient. In addition, the |
| | | dentist can contact, text and |
| | | request 24-hour access to the |
| | | patient EDR before of during |
| | | the tele appointment. |
| | | The patient showed receive a |
| | | notification and text message |
| | | for approval. |
| | Scheduled: | The above feature will be the |
| | Clinic | same in clinic section too |
| | | with adding the type of |
| | | planned appointment. |
| | In current: | Like an Excel sheet that |
| | | dentist is able to choose |
| | | according to (patient name |
| | | list, treatment code, |
| | | diagnosis, age, gender, |
| | | scheduled: in tele or 2^{nd} |
| | | opinion or in clinic) |
| | | Once the dentist press in the |
| | | Patient's name: |
| | | (name/gender/DOB/zip code |
| | | and app code's, a history of |
| | | Dental clinical notes, |
| | | diagnosis, treatment code - |
| | | contact made by tele or 2^{nd} |
| | | opinion or in clinic) in all |
| | | can contact texted or phone |
| | | & access to their HER. |
| | | Patient all EDR information |
| Marketing | Packages Analysis | The dentist can subscribe to |
| | ODONTYN Schedule | specific marketing packages, |
| | Analysis. | each package contains |
| | | features and cost. The |
| | | packages been prescribed in |
| | | section 2.1.7. |
| | | Options: |
| | | on general/gender/or |
| | | location/age |
| | | based on which kind of |
| | | schedule (Urgent tele/ |
| | | Urgent clinic/non urgent |
| | | 2nd option, non-urgent |
| | | clinic) |
| | | Patient Satisfaction post |
| | | schedule |
| | | Dentist efficient in reporting |
| | | the EDR post |
| | | schedule/appointments (time, |
| | | full data, share with patient, |
| | | referral) |
| | | Finance. |
| | ODONTYN public | An automatic analysis of |
| | review | how many times the patient |
| | analysis | access to their account, like |
| | | and rate. |
| | | Patient access based on |
| | | general/gender/or |
| | | location/age |
| | | Patient Likes based on |
| | | general/gender/or |
| | | location/age |
| | | Patient comments based |
| | | on general/gender/or |
| | | location/age. |
| | ODONTYN clinic | An automatic analysis of the |
| | analysis | number of patient list, ages, |
| | | genders, zip code and |
| | | treatment provided. |
| | Social Media | Analysis feature of the |
| | Analyses | comments rating, services |
| | | and schedule that the patient |
| | | is receiving form the app. |
| | | Once the social media |
| | | platform in linked to this |
| | | app, over all analysis of each |
| | | app can be illustrated as this |
| | | will help the dentist to know |
| | | where is more effective for |
| | | him to invest on marketing. |
| In my Patient | | The dentist will have access |
| list → Current | | to 7 features of the patient |
| section (save | | EDR (upload dental, my |
| patients and | | teeth chart, Gum chart, extra |
| patient who | | oral examination, EDR |
| allowed to | | history, Medical record, |
| share their | | medication, x ray, digital |
| EDR access to | | impression, pictures) |
| the dentist) | | Side icon where unable the |
| | | dentist to save the patient |
| | | EDR into their system. |
| | | Dentist will have the access |
| | | for only 24 hours to check |
| | | and update. The history of |
| | | dentist editing should be |
| | | saved for any breach |
| | | reference. |
| | Upload EDR - | The dentist can syn their |
| | in Patient | EHR to this app or download |
| | version | manually in a specific form. |
| | | Natural language processing |
| | | to summary old EDRs. |
| | | Dentist once press on. “+”, |
| | | he/she can add new |
| | | examination by selecting |
| | | teeth number in the chart and |
| | | right it down, examination |
| | | carried out in open box, |
| | | select the ICD 10 Dental |
| | | diagnosis codes and select |
| | | treatment plan code. |
| | | A color coding with three |
| | | options that dentist needs to |
| | | select; completed, on hold, |
| | | planned. Day and time need |
| | | to appear at the end of the |
| | | submission. The form |
| | | automatically added to: |
| | | uploaded Dental, My teeth, |
| | | and EDR features. |
| | dental chart → | The dentist can syn their |
| | in patient | EHR to this app or download |
| | version | manually in a specific form. |
| | | Same as above save |
| | | automatically in different |
| | My gum → | The dentist can syn their |
| | in patient | EHR to this app or download |
| | version | manually in a specific form. |
| | | Color code (completed, on |
| | | hold, planned) that the |
| | | patient needs to select prior |
| | | to submission |
| | Update Extra oral | As it been explained it |
| | examination-patient | divided into four sections. |
| | version | Dentist can edit all four by |
| | | clicking in “+” |
| | EDR → patient | Same as a feature “upload |
| | version | EDR - inpatient verson” |
| | Medical record. → | The ability of dentist to edit |
| | patient | vital signs and BMI |
| | version | measures. |
| | | The ability to the dentist to |
| | | add a medical condition with |
| | | the diagnosed year. |
| | | Color system coding that the |
| | | dentist has to select; green as |
| | | a complete, yellow as active |
| | | and red as a uncontrollable. |
| | | Once the dentist submits, his |
| | | DDW code appear next to |
| | | the editing. |
| | Medication → | The dentist has the ability to |
| | patient | edit in the three sections; |
| | version | currently taking, recently |
| | | prescribed and allergies. |
| | | In section 1 and 2; the dentist |
| | | has to write; the medication |
| | | list, dosage, instruction, code |
| | | of provider and select one of |
| | | the color coding (green in |
| | | need, yellow temporary and |
| | | red complete.) |
| | X-rays → Patient | Dentist can upload DICOM |
| | version | of GIP image of the patient |
| | | and select the type the |
| | | patient wishes to save it as |
| | | their account. The time and |
| | | date and provider DDW code |
| | | will be there. |
| | | The dentist can syn their |
| | | EHR to this app or download |
| | | manually by uploading |
| | | DICOM or GIP. |
| | Digital impression → | Similar to “Medication to |
| | patient | patient version”, except |
| | version | between STL file or GIP. |
| | Pictures → Patient | Upload intraoral pictures in |
| | version | templet taking from smart |
| | | phone camera: |
| | | Front view of the patient in |
| | | relaxed |
| | | Frontal view of the patient |
| | | smiles socially |
| | | Frontal view smile maximum |
| | | Side view of the patient |
| | | relaxed |
| | | Side view of patient smile. |
| | | Close up smile photo |
| | | Extraoral picture retractre |
| | | lips full profile in MIP |
| | | Extraoral picture retractre |
| | | lips full profile in slight |
| | | opening |
| | | Extraoral picture Retract lip |
| | | upper teeth (with back |
| | | separation) |
| | | Intra oral retracting in MIP, |
| | | protrusion, lateral excursion. |
| | | Intra oral picture occlusal |
| | | upper |
| | | Intra oral picture occlusal |
| | | Lower |
| | Smile design | Once the dentist selects the |
| | | image; feature appears below |
| | | the image: |
| | | Shapes to use some shapes |
| | | to aid in analysis, draw and |
| | | erase feature, draw with tack |
| | | on or off, and smart design. |
| | | [symbol] |
| | | Dentist can select the type of |
| | | teeth shape to help the |
| | | patient to visualize his |
| | | analysis. |
| | | The dentist can save and |
| | | unsaved the image in dentist |
| | | ODONTYN view only. |
| Prescribe | direct to patient's | The dentist can prescribe |
| medication | zip code | mediation online, select the |
| | closet pharmacy | mediation name, dosage, |
| | | instruction. |
| | | The dentist can refill the |
| | | prescription, once the patient |
| | | requests it. |
| | | The prescription can be |
| | | linked to the patient zip code |
| | | nearest pharmacy. |
| Feedback and | analysis with | In two sections: feedback on |
| consent | set questions | hold, create a feedback. |
| forms | from the app | The dentist can fill and |
| | | evaluate the app through |
| | | filling a feedback form. |
| | | By filling the 6-month |
| | | feedback form the dentist |
| | | will get points and benefits. |
| Contact | communicate directly | The dentist can select the |
| insurance: | with the insurance | name of insurance and |
| | (send pre-estimation | contact directly through |
| | and claims) | phone a call or email. |
| | | The dentist can communicate |
| | | directly with the insurance |
| | | and sending pre-estimation |
| | | and claims. |
| | | The dentist can keep track on |
| | | a requested estimation. |
| My | | This feature an provide a |
| finances | | future analysis of what kind |
| | | of treatment that the dentist |
| | | is mostly gaining from, the |
| | | amount of patient pool |
| | | increases through this app. |
| | | The amount of the marketing |
| | | investment and returns. |
| Clinic | (check in/check | The patient checks in upon |
| management | out . . . , thank | arrival, the system will |
| (optional) | you for visit) | automatically be linked to |
| | | the patient clinic. |
| | | Patient once from all his |
| | | record have been shared with |
| | | him/her, they can check out. |
| | | Upon check out an automatic |
| | | thank you message from the |
| | | clinic and reminder to the |
| | | patient to rate and leave a |
| | | comment. |
| | | An additional device can be |
| | | insulted in the clinic door |
| | | iterance, that automatically |
| | | thank the patient and remind |
| | | them to rate and comment. |
| Evidenced | (future upgrade) | Search icon, that can lead |
| based | | the dentist directly to |
| search | | evidence-based dentistry in |
| | | PubMed and dental known |
| | | Journals. |
| Dental | (future upgrade) | Dentist can sell their |
| products | | products or buy products |
| | | from companies |
| | | Once the dental company's |
| | | (ex; Ivoclar Vivadent, |
| | | Straumann, Nobel care) |
| | | collaborate, they can sell |
| | | their products in this app. |
| | | The dentist can request and |
| | | order directly from the |
| | | companies. |
| | | Upon dental company |
| | | collaborations, dentist can |
| | | receive 5% discount after the |
| | | reach to a specific award |
| | | level. |
| Continue | | The app can be linked to an |
| Education | | international known |
| | | organization that provide |
| | | annual conferences, |
| | | seminars, webinars and |
| | | workshops. |
| | | It can be used as a marketing |
| | | for those in dental app |
| | | platform. |
| | | The dentist can register |
| | | directly through this app. |
| My calendar | | A calendar appear with list |
| | | of patient schedule and the |
| | | type of the appointment |
| | | (green non urgent/2^{nd} |
| | | opinion/red urgent) once the |
| | | dentist click on the link |
| | | dentist can request (the |
| | | patient)to access to their |
| | | calendar for 24 hours. Box of |
| | | presenting complain, and |
| | | patient comment appear. |
| Multidisci- | | A search referral list appears |
| plinary | | in the top of the screen that |
| team | | allow the dentist to filter |
| | | their search (dental specialty, |
| | | Dentist ODONTYN code or |
| | | name (optional, zip code, |
| | | patient preferred language, |
| | | type of insurance coverage). |
| | | The dentist can save his |
| | | referral to his list. |

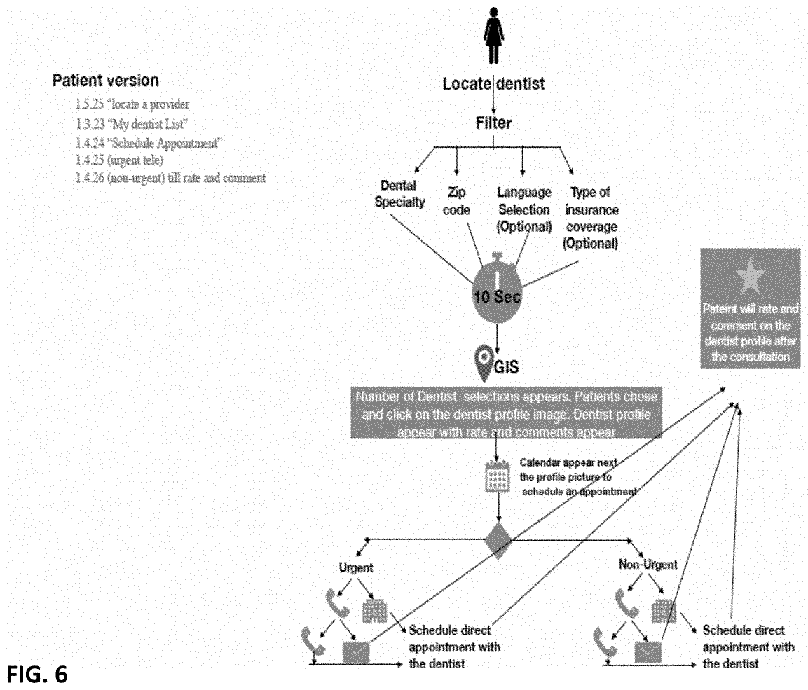

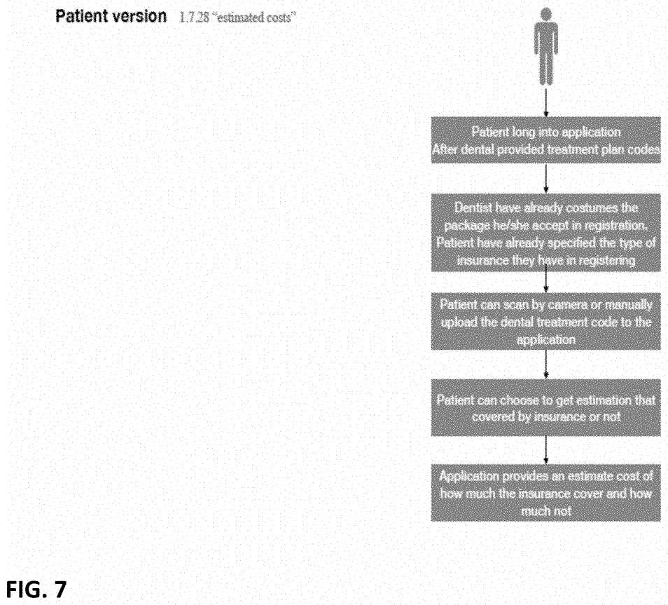

FIGS. 6-11 illustrate a detailed interaction flow/wireframe of the list sequences of user actions and system responses. FIG. 6 is a flow diagram illustrating five main features “locate a provider,” “my dentist list,” schedule appointment, “urgent tele-dentistry,” and “non-urgent dentistry” provided by the patient platform. FIG. 7 is a process flow for “estimated costs” section performed by the patient platform. The method includes the steps of patient login into application after dentist provided treatment plan codes (step 1), dentist accepting the package in registration and patients specifying the type of insurance they have in registering (step 2), patient scanning by camera or manually and uploading the dental treatment code to the application (step 3), patient can choose to get an estimate/inquiry as to insurance coverage (step 4), and application provides an estimate cost of how much the insurance cover and how much is not covered (step 5).

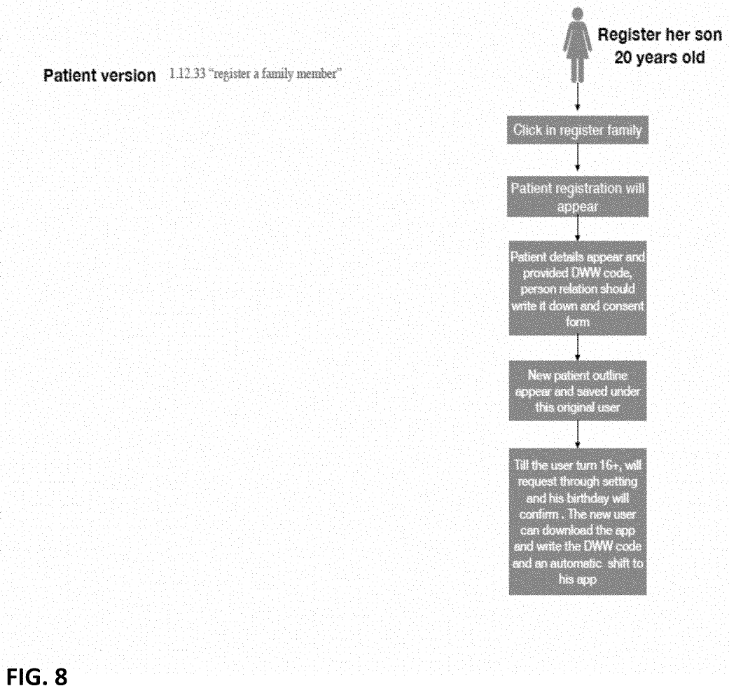

A flow diagram showing the method of registering a family member is illustrated in FIG. 8, according to which a patient can register a family member by making a selection of “register family member” (step 1), patient registration will appear (step 2), patient details will appear and provided with DWW code and consent form (step 3), a new patient outline will appear and will be saved under the original user (step 4) and then after age verification, a user can download the application and enter the DWW code (step 5).

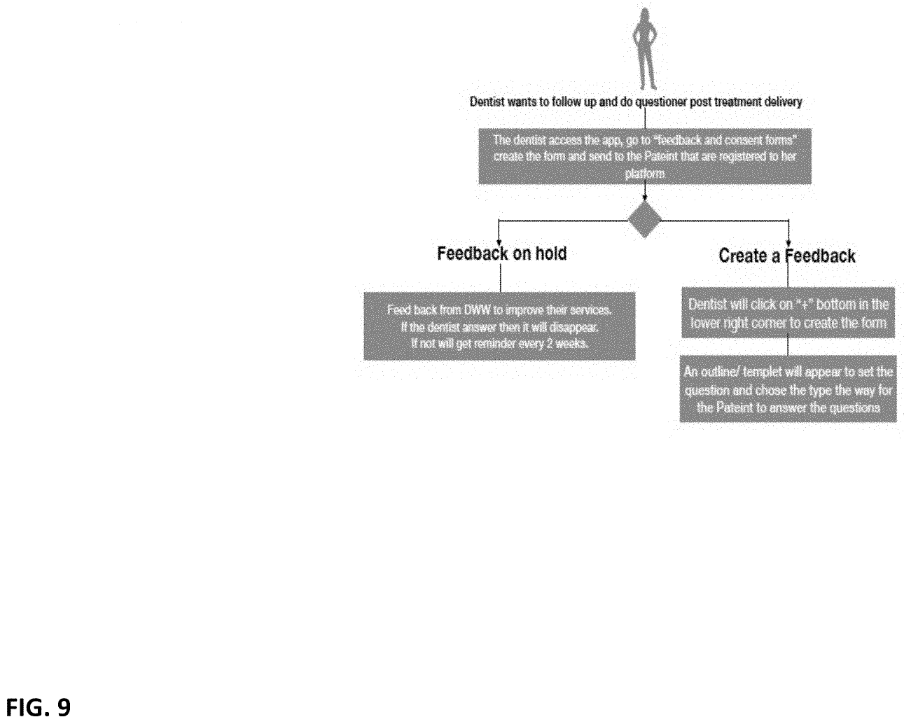

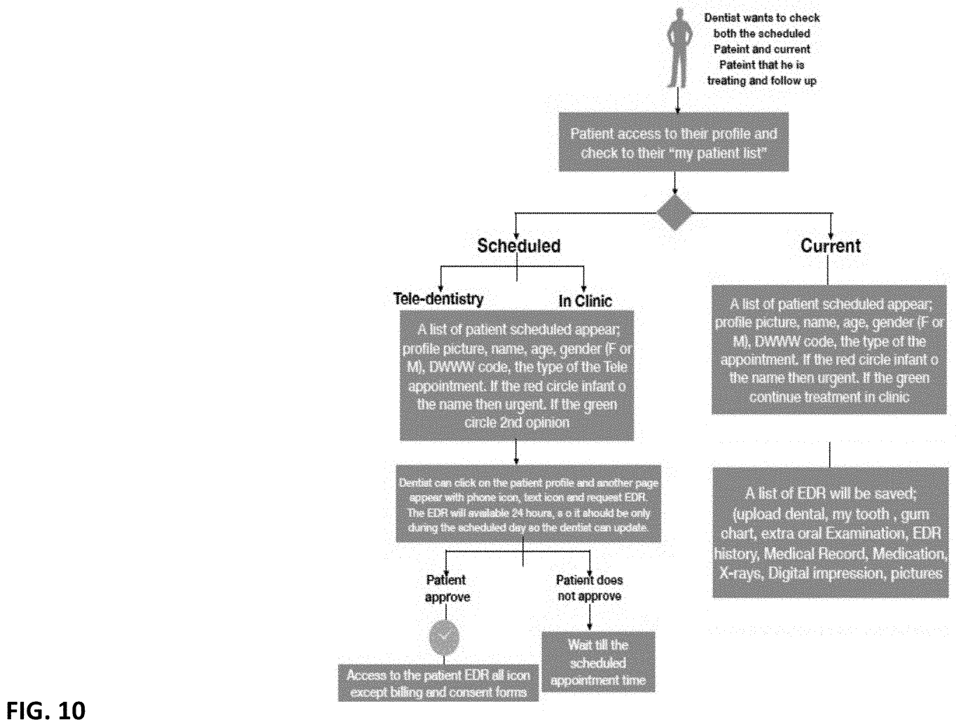

FIG. 9 is a flow chart illustrating a process flow for “my patient list”, “scheduled (urgent/2^{nd} opinion) and “in current” features performed by the dentist platform when the dentist wants to check both the scheduled patient and current patient that he is treating and following up. FIG. 10 is an illustration of the process flow for the “feedback and consent forms” section of the dentist platform allowing the dentist to follow up and perform a questioner post treatment delivery.

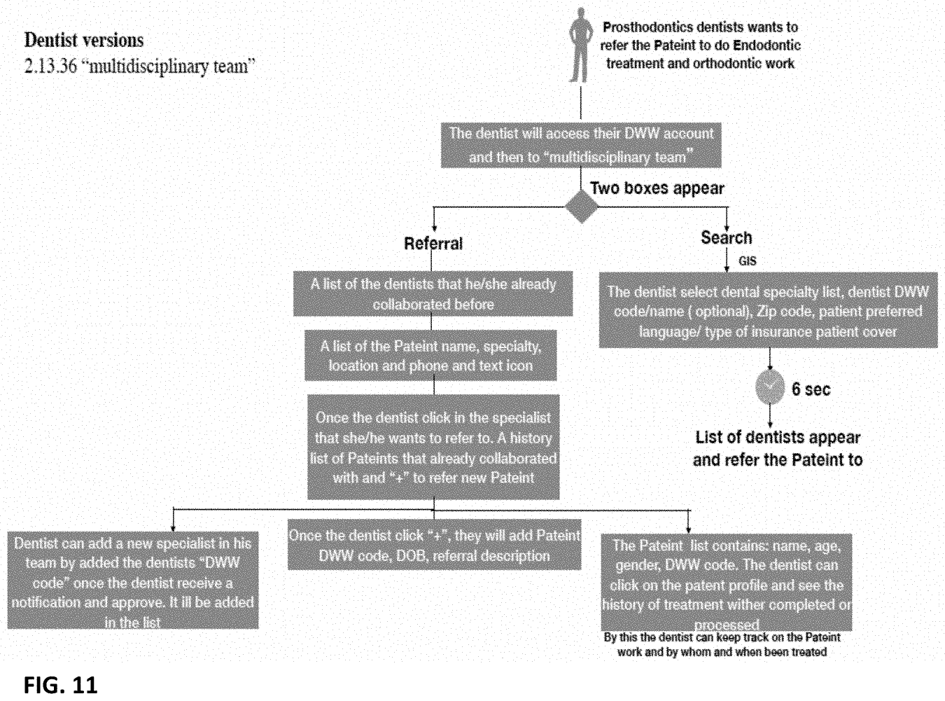

FIG. 11 shows the “multidisciplinary team” feature for referring patients for endodontic treatment and orthodontic work.

The system of the present invention is configured to extract medical and dental records using APIs created by health care providers. Once the medical records are extracted from a database, the system is further configured to arrange the records into simplified templates for ease of use by both dentists and patients utilizing machine learning and language processing methods. The system of the present invention is configured to create a dental record database that can be used in dental clinics and accessible by the patients and dentists at any time and place, through computers or smart mobile devices. The dental record is in the user patient friendly format and summarize the main information for the dentist to provide proper consultations through tele-dentistry or in-clinic.

The system of the present invention is configured to allow the patients to request Dental and/or medical records from the patients' previous clinic, wherein the system will send an email and reminders to the requested clinic. Once the clinic sends the dental record, the system of the present invention will automatically summarize and organize the dental record into friendly easy looking ODR (Odonto Dental Record). If the clinic or the dentist registered within the system of the presnet invention and have an integratabtle software, then the dentist will activate an exiting software to be used by the system, and the data will be automatically transferred to ODR format. This process will carry out through API and programming codes. The data will be automatically updated.

If the clinic or the dentist is not registered or does not have an integrable software or use hard copies. Then the clinic/dentist will send the record to the system of the present invention, which is configured to develop an AI/natural processing software-implemented method to reorganize the data into the ODR. If the record has been sent in a pdf form, then PDF dental record will be saved in the patient's profile. Patients will be able to share their ODR to their new dentist with a click of the button. The dentist can access and review the patient's ODR for 24 hours. The dentist can update the information too within the 24 hrs.

With reference to the “My Dental Profile” section, the system is configured to provide the patient with the ability to upload PDF EDR, and it will be saved into the system. The natural processing feature will be carried out to select the primary information displayed in the summary table, such as the date of the procedure, tooth side, treatment code. The patient will see the table summary that describes the workflow of the leading dental work carried out.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. A system for managing electronic medical records and medical record information and for providing a communication platform for medical providers and patients comprising:
a patient platform interlinked with a dentist platform by a communication interlinking platform configured to communicate between the patient and dentist platforms through a communication network.

2. The system of claim 1, wherein the patient platform is a mobile application adapted for use on a smart phone.

3. The system of claim 1, wherein the dentist platform is a website accessible through computers and smart mobile devices.

4. The system of claim 1, wherein the dentist platform is a mobile application.

5. The system of claim 1, wherein the system is configured to be HIPPA compliant, secure and capable of protecting user confidentiality.

6. The system of claim 1, wherein the patient platform is configured to have a plurality of functionalities provided for each patient.

7. The system of claim 1, wherein the dentist platform is configured to have a plurality of functionalities provided for each dentist.

8. The system of claim 1, further comprising a first module configured to extract medical and dental records using APIs created by health care providers and to arrange the extracted records into simplified templates for ease of use by both dentists and patients utilizing machine learning and language processing methods.

9. The system of claim 1, further comprising a second module configured to create a dental record database that can be used in dental clinics and accessible by the patients and dentists at any time and place, through computers or smart mobile devices or both.

10. The system of claim 9, wherein the dental record is in the user patient friendly format summarizing the main information for a dentist user to provide proper consultations through tele-dentistry or in-clinic.
